(19) United States
(12) Patent Application Publication
(10) Pub. No.: US 2013/0035576 A1
(43) Pub. Date: Feb. 7, 2013

(54) SYSTEM AND METHOD FOR MAPPING GASTRO-INTESTINAL ELECTRICAL ACTIVITY

(71) Applicants: Gregory Brian O'Grady, Auckland (NZ); Leo Koon-Wah Cheng, Auckland (NZ); Peng Du, Auckland (NZ); Jonathan Christopher Erickson, Lexington, VA (US); Path Lessop-Pullan, Epsom (NZ)

(72) Inventors: Gregory Brian O'Grady, Auckland (NZ); Leo Koon-Wah Cheng, Auckland (NZ); Andrew John Pullan, Auckland (NZ); Peng Du, Auckland (NZ); Jonathan Christopher Erickson, Lexington, VA (US)

(73) Assignee: AUCKLAND UNISERVICES LIMITED, Auckland (NZ)

(21) Appl. No.: 13/391,621

(22) PCT Filed: Aug. 23, 2010
(86) PCT No.: PCT/NZ2010/000164

§ 371 Date: Oct. 10, 2012

(30) Foreign Application Priority Data
Aug. 21, 2009 (NZ) 579235

(51) Int. Cl. A61B 5/0492

(52) U.S. Cl. 600/373

(57) ABSTRACT

A gastro-electrical activity mapping system and comprises a catheter insertable through a natural orifice into the gastro-intestinal (GI) tract and comprising an array of electrodes for contacting an interior surface of a section of the GI tract to detect electrical potentials at multiple electrodes, and a signal analysis and mapping system arranged to receive and process electrical signals from multiple electrodes of the array and spatially map GI smooth muscle electrical activity as an activation time map, a velocity map, or an amplitude map, which may be in the form of contour plots and may be mapped on an anatomical computer model of at least the section of the GI tract and may be animated. A GI mapping method and catheter are also claimed.

### FIELD OF INVENTION

The invention relates to a system and method for mapping gastro-intestinal electrical activity.

### BACKGROUND

Gastric dysrhythmias underlie or contribute to diseases including gastroparesis, functional dyspepsia, and gastro-esophageal reflux disease (GERD). Gastroparesis is a condition in which the stomach typically fails to empty properly after a meal, leading to symptoms of early fullness, bloating, pain, nausea, vomiting and malnutrition and possibly death in severe cases. Medical guidelines suggest that the majority of patients with suspected gastroparesis should receive an upper gastrointestinal (GI) endoscopy study (a video-guided examination of the inside of the stomach). Functional dyspepsia is a condition characterised by symptoms of ‘chronic indigestion’ lasting at least weeks to months, which may include bloating, nausea, and pain after eating. The causes of functional dyspepsia are not well understood, however dysrhythmic gastric activity has been clearly implicated, with up to 60% of adult dyspeptic patients showing abnormal gastric electrical activity. Delayed gastric emptying occurs in 25-40% of functional dyspepsia. Upper GI endoscopy is a standard diagnostic tool for assessing patients presenting with dyspepsia. Delayed gastric emptying also affects a significant sub-population of patients with GERD, and gastric dysrhythmia has been implicated.

Peristaltic activity in the GI tract is coordinated by a propagating electrical activity termed slow waves. GI slow waves are initiated and spread via networks of interstitial cells of Cajal (ICCs), which are coupled to the smooth muscle layers in the GI tract wall. In the human stomach, slow waves originate at a pacemaker site high on the greater curvature, and propagate toward the antrum at a normal frequency of approximately three cycles per minute.

Electrocardiography (ECG) is a routine diagnostic test for cardiac dysrhythmias, in which electrodes are placed on the skin to record the distant organ electrical activity. Electrogastrography (EGG) or the assessment of GI electrical activity through skin electrodes has also been proposed but despite research efforts has failed to meet clinical expectations, partly because the quality of GI electrical signals recorded at the skin is too limited to provide accurate diagnostic value. Also, EGG is a summation of all of the electrical activity occurring in the stomach and so cannot provide accurate information regarding the normal or abnormal propagation of the individual slow wave cycles.

A SQUID (Super Quantum Interference Device) can be used to measure the magnetic fields associated with GI electrical activity, but is a multi-million dollar device that must also be housed in a magnetically-shielded room, and analysis of the signals obtained is complex and has not yet been reliably achieved. Also, the resolution achieved via a SQUID may be suboptimal.

A roving electrode placed into sequential sites on the mucosa of the stomach, or a small number of electrodes linearly arranged and attached to a naso-gastric tube, can give some indication of GI dysrhythmic activity, however may not reliably provide information on the spatial propagation of gastric slow wave activity and therefore cannot describe abnormal velocities, propagation directions, or dysrhythmias accurately.

High-resolution mapping of GI electrical activity by measurement at the serosal surface requires invasive surgical access and therefore is not appropriate for use in the vast majority of patients with gastrointestinal symptoms.

### SUMMARY OF INVENTION

In broad terms in one aspect the invention comprises a system for mapping gastro-electrical activity comprising:
a catheter insertable through a natural orifice into the gastro-intestinal (GI) tract and comprising an array of electrodes for contacting an interior surface of a section of the GI tract to detect electrical potentials at multiple electrodes,
a processing system arranged to receive and process electrical signals from multiple electrodes of the array and spatially map the GI smooth muscle electrical activity at said section of the GI tract.

In some embodiments the system is arranged to visually display a map or animation of GI electrical activity in real time.

In some embodiments the system is arranged to display any one or more of an activation time map indicative of the propagation of electrical activity, a propagating wavefront animation, a velocity map indicative of slow wave velocity and/or direction, an amplitude map of slow wave signal amplitudes across the stomach, and a dysrhythmia map of the GI electrical activity.

In some embodiments the system is arranged to map the GI electrical activity on to a generic or a subject-specific anatomical model of the section of the GI tract.

In some embodiments the system may be arranged to determine or approximate the relative locations of electrodes of the array in contact with the interior surface of the section of the GI tract, to develop or modify an anatomical model of the section of the GI tract, and to map the GI smooth muscle electrical activity onto the anatomical model.

In some embodiments the system may comprise a reference database indicative of geometries of one or more sections of the GI tract and related characteristics such as subject height and sex relating to each geometry, and the system is arranged to select a best-fit geometry from the database for each subject under study and optionally modify the selected geometry.

In broad terms in a further aspect the invention comprises a method for mapping GI electrical activity which comprises inserting a catheter through a natural orifice into the GI tract and causing an array of electrodes of the catheter to contact an interior surface of a section of the GI tract to detect electrical potentials at multiple electrodes, and receiving and spatially mapping from the electrical signals GI electrical activity at said section of the GI tract.

In a preferred form said processing of the electrical potential signals detected at the electrodes includes amplifying and/or filtering the signals, identifying slow waves, and animating the individual propagating waves over a generic or subject-specific anatomical model.

The processing may also include making time activation maps of waves, calculating velocity and amplitude fields from the activation maps, and displaying the activation maps and velocity fields over the anatomical model.

The processing may also include quantifying averages of any one or more of propagation directions, normal versus abnormal propagation, types of dysrhythmias, frequencies, regional stomach velocities, amplitudes, and reporting average figures and/or representing an average map of a recording period.

The processing may also include comparing the GI electrical activity to a stored reference database to provide an indication of normal or abnormal GI electrical activity.

In broad terms in a further aspect the invention comprises a catheter for mapping GI electrical activity, insertable through a natural orifice into the GI tract and comprising an array of sufficient electrodes arranged to contact around and/or along an interior surface of a section of the GI tract to detect electrical potentials to enable mapping of electrical activity at said section of the GI tract.

In some forms the catheter comprises an inflatable or otherwise expandable electrode carrier such as a balloon or expandable mesh, carrying on an exterior surface the array of electrodes, the electrode carrier being inflatable or expandable via the catheter when in place to cause electrodes to contact the interior surface of the GI tract. The catheter may also comprise a tube or other element that extends internally towards the distal end of the catheter to assist in locating the catheter in the desired location in the GI tract.

The invention includes an inflatable or expandable balloon or mesh or other attachable electrode carrier end for a catheter for mapping GI electrical activity, attachable to an end of the catheter, and inflatable or expandable through the catheter when in place, the catheter end comprising the array of electrodes for contacting the interior surface of the GI tract.

The system and method of the invention are intended to be useful in the diagnosis of gastric dysrhythmias including in gastroparesis and functional dyspepsia, and may also be useful in the diagnosis of disease mechanisms in gastro-oesophageal reflux disease and other gastro-intestinal motility disorders such as small intestinal, colonic and rectal dysmotility disorders, or in other smooth-muscle-lined viscera, including the bladder.

The system of the invention may be employed as an adjunct to upper or lower GI endoscopy.

The system and method of the invention may be useful to guide therapies for gastric dysmotility disorders, including gastric electrical stimulation, targeted ablation of aberrant conduction pathways and targeted drug delivery.

In broad terms in a further aspect the invention comprises a method for detecting GI slow wave activations in GI electrical activity which includes analysing the GI electrical activity for events indicative of GI slow waves and clustering detected events into groups each relating to a common slow wave based on temporal closeness.

In broad terms in a further aspect the invention comprises a method for clustering detected GI slow wave events in GI electrical activity into groups each relating to a common slow wave based on temporal closeness, which comprises clustering detected events by a region growing using polynomial surface estimate stabilization method.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

### BRIEF DESCRIPTION OF THE FIGURES

Embodiments of the invention are further described with reference to the accompanying figures, without intending to be limiting, in which:

FIG. 1 shows one embodiment of a gastro-intestinal (GI) mapping catheter, unexpanded,

FIG. 2 shows the GI mapping catheter of FIG. 1, expanded,

FIG. 3 schematically shows intubation of the GI mapping catheter of FIGS. 1 and 2, into the gastric antrum,

FIG. 4 shows the GI mapping catheter of FIGS. 1 and 2 after intubation and expansion until the electrode array of the mapping catheter contacts the mucosal surface of the gastric antrum,

FIG. 5 shows another embodiment of a GI mapping catheter in position in the gastric antrum,

FIG. 6 shows a further embodiment of a GI mapping catheter in position in the gastric antrum,

FIG. 7 shows the GI mapping catheter of FIG. 6 unexpanded,

FIGS. 8a-c shows a recoil spring system for the electrodes of a GI mapping catheter of the invention,

FIG. 9 shows an example of a user-display on a VDU presented by an EGG system of the invention,

FIG. 10 shows another example of a user-display including actuation time and velocity maps of GI electrical activity, presented by a GI mapping system of the invention,

FIGS. 11a and 11b show further including actuation time and velocity maps of GI electrical activity, on a stomach model,

FIG. 12 is a flow chart illustrating signal analysis, mapping, and model fitting stages of a preferred embodiment GI mapping system and method of the invention,

FIG. 13 flow chart of a preferred embodiment method for GI slow wave activation time identification,

FIG. 14 is a flow chart of a preferred embodiment clustering method for clustering or partitioning of activation times into separate gastric slow wave groups,

FIG. 15a is a pixelated isochronal activation time map or a part thereof and FIG. 15b shows such a smooth filled contour activation time map with isochronal lines,

FIG. 16 shows an isochronal activation time map and a velocity map,

FIGS. 17a and 17b show GI slow wave amplitude and velocity respectively in different gastric regions (normal human population),

FIG. 18 shows one electrode channel of GI slow wave data recorded from the serosal surface of the GI tract, referred to in the subsequent description of experimental work,

FIG. 19 shows two channels of GI slow wave activity and stimulation artifact recordings from the mucosal gastric surface, as referred to in the subsequent description of experimental work,

FIG. 20 shows GI slow wave activity from three electrodes, referred to in the subsequent description of experimental work,

FIGS. 21a and b show a spatial activation maps from two mucosal recordings of consecutive GI slow waves, referred to in the subsequent description of experimental work,

FIG. 22 shows the points at which the stomach was measured during surgery to reconfigure a anatomical model to be subject specific, referred to in the subsequent description of experimental work, and

FIGS. 23 to 26 show activation time and velocity maps of gastric electrical activity, referred to in the subsequent description of experimental work.

### DETAILED DESCRIPTION OF EMBODIMENTS

GI Mapping Catheter

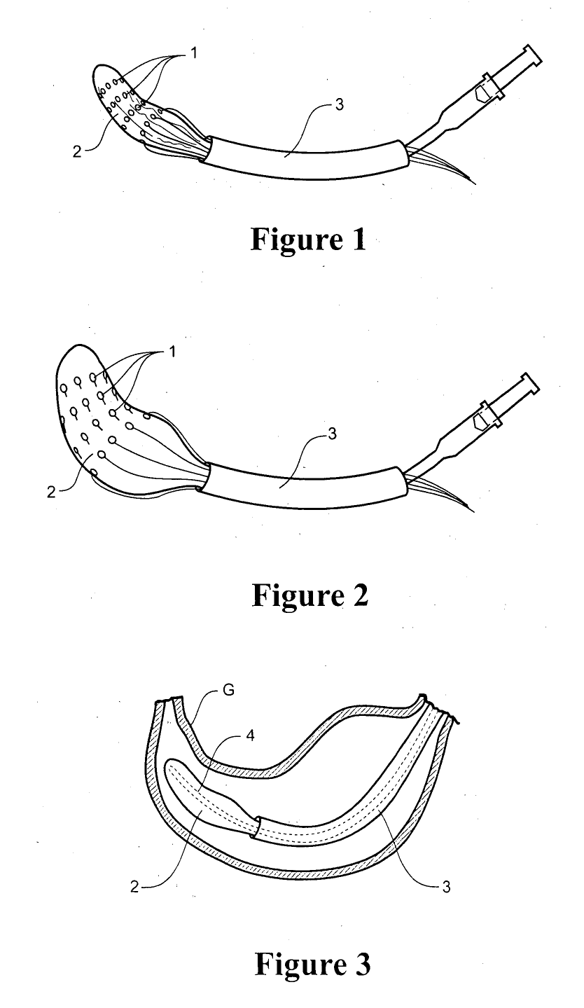

FIGS. 1 and 2 show one form of a mapping catheter useful for mapping GI electrical activity. The catheter comprises an array of electrodes some indicated at 1 spaced around an expandable electrode carrier comprising an inflatable balloon 2, attached to a nasogastric or oral gastric or similar tube 3. Signal wires or conductors (electrically insulated) one from each electrode 1 pass through the tube 3 from the catheter to exit the proximal end of the nasogastric tube, for example at a plug for coupling the signal lines to electronic instrumentation. FIG. 1 shows the balloon electrode carrier 2 deflated and FIG. 2 shows it inflated.

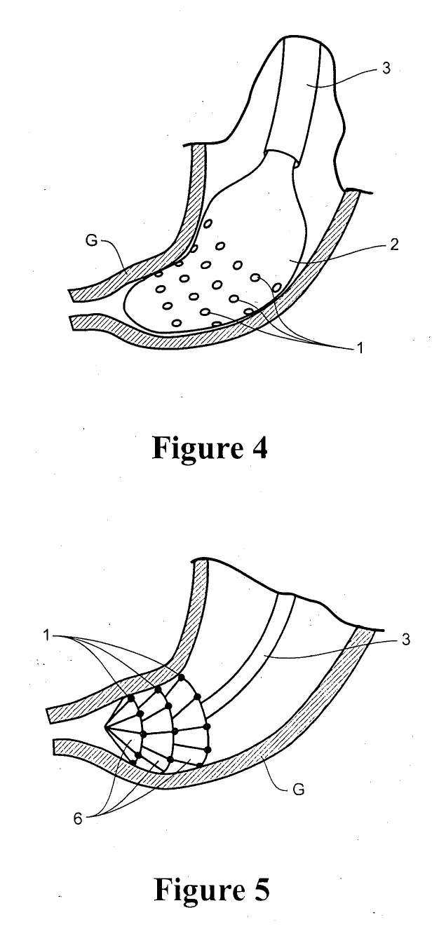

In use the catheter with the balloon 2 deflated is intubated temporarily via a natural orifice, such as via the mouth, into the GI tract and when in position at the desired location, such as in the gastric antrum, gastric corpus, upper small bowel, rectum, large bowel, or bladder, is expanded by inflation through the lumen of the tube 3 until the electrodes 1 or at least some electrodes contact the mucosal surface that part of the GI tract. The catheter may also comprise a second internal catheter tube (which may alternatively serve for inflation of the balloon) or other element that extends through the tube 3 to within the balloon 2, as indicated at 4 in phantom outline in FIG. 3, to assist in locating the tip of the balloon in the desired position. FIG. 3 shows the GI mapping catheter positioned in the gastric antrum indicated at G and before inflation, and FIG. 4 shows the catheter after inflation to cause multiple electrodes 1 to contact the mucosal surface around the interior of and spaced lengthwise of the GI tract, sufficient to obtain electrical potentials indicative of GI electrical activity around and lengthwise of that part of the tract. The electrodes are preferably but not exclusively point electrodes, such as convex pointing electrodes, which at least when the balloon 2 is inflated stand perpendicular to the surface of the balloon, such that they indent the mucosa to enhance contact and signal quality.

FIG. 5 shows an alternative form of catheter which comprises multiple fold-out resilient electrode carrying elements such as metal wires 6 from around the catheter end, the ends of which carry or comprise the electrodes 1. At insertion the elements 6 are retained folded tightly against the end of the catheter against their natural resilience for example by an external cover (not shown) which can be drawn back up the catheter remotely after positioning of the catheter, to allow the resilient electrode carrying elements to spring or fold out to-push the electrodes 1 against the mucosal surface, again around and lengthwise of that part of the GI tract. The fold out elements 6 may optionally be ordered in series of circular rows around and spaced along the catheter end, which may be connected so that each row in use folds out like an umbrella, or the elements may be otherwise regularly (or irregularly) spaced around and along the catheter end.

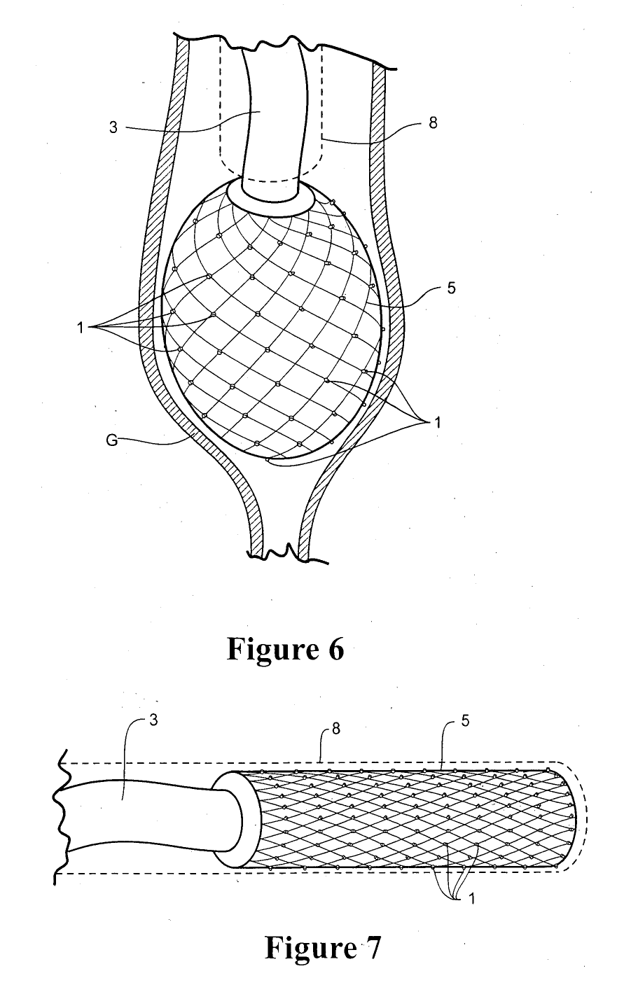

FIGS. 6 and 7 show a further alternative form of GI mapping catheter comprising an expandable mesh 5, carrying a similar array of spaced electrodes some indicated at 1. The catheter mesh 5 may be formed of a resilient plastics material or a spring metal such as surgical grade stainless steel, and having a memory for its expanded position, which is mechanically restrained unexpanded as shown in FIG. 7 until in position within the GI tract for example by a covering sleeve 8 which can then be withdrawn remotely by the clinician to the position of the sleeve shown in FIG. 6 to allow the mesh catheter to resiliently expand as shown in FIG. 6 to press the catheter electrodes against the interior of the GI tract. In this and all embodiments expansion, and contraction for withdrawal, of the catheter may be initiated or controlled by a trigger or other device on a handle or control, to which the catheter is connected by the tube 3, through which one or more control lines or similar pass to the catheter and/or surrounding sleeve. FIG. 6 shows the catheter in position in the gastric antrum and FIG. 7 shows the catheter unexpanded and within contraction sleeve 8. Rows or another array of electrodes 1 are spaced around and lengthwise of the expandable mesh 5. As before the electrodes 1 are preferably point electrodes, which at least when the catheter is expanded stand perpendicular to the catheter surface, to indent the mucosa to enhance contact and signal quality. The electrodes 1 may be carried by the mesh 5 so that during intubation of the catheter the electrodes lay against or adjacent the catheter mesh and after the catheter has been positioned in the desired part of the GI tract, may be caused to move to protrude outwardly from the carrier mesh, to press against the gastric mucosa. The sleeve 8 typically in the form of a sock of a relatively rigid plastics material and as long as the catheter itself, surrounds the catheter when the catheter is unexpanded so that the catheter is contained within the sleeve. The catheter may be intubated to the desired position unexpanded as shown in FIG. 7 then before expansion of the mesh catheter (or inflation of a balloon catheter), or after only partial expansion of the catheter, folding or pivoting electrodes 1 may be caused to move to their protruding or contact position following which the sleeve 8 may be withdrawn so that the catheter is caused to expand fully to cause the outwardly facing electrodes to then contact the interior of the GI tract.

In yet another embodiment a mesh catheter such as described in relation to FIGS. 6 and 7 may comprise a balloon within, which is inflated in use to expand the mesh catheter and press the electrodes against the mucosal surface. Electrodes 1 may each be mounted for reciprocal protrude-withdraw movement within a small outwardly facing cylinder carried by the mesh, so that full expansion of the balloon within the mesh will both expand the mesh fully and also push the electrodes from within the mesh to protrude. Each electrode mounting may comprise a small recoil spring arranged to withdraw the electrode when the balloon is deflated for withdrawal of the catheter from the patient.

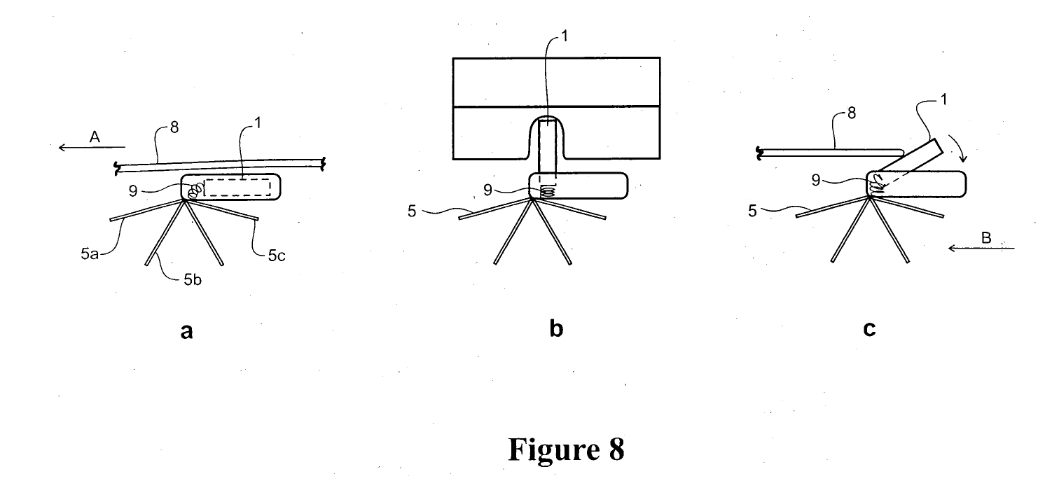

FIGS. 8a-c show a single electrode 1, which is mounted to the catheter 5 via a small coil spring 9. In the example shown the catheter is a mesh catheter as previously described and each or many electrodes may be mounted individually at intersections of individual mesh elements 5a-5c (as are other electrodes of the catheter—only one being shown in FIG. 8). When the catheter is within the sleeve 8 each electrode 1 is bent over as shown in FIG. 8a against the mesh, allowed by the spring mounting described. When the catheter within the sleeve has been intubated to the desired position within the GI tract and the sleeve is withdrawn sufficiently i.e. the sleeve 8 moves in the direction of arrow A in FIG. 8a, the electrodes 1 stand up perpendicular to the mesh 5 and press against the mucosa, as shown in FIG. 8b. The springs 9 are sufficiently strong and resilient to cause the electrodes to so move. Subsequently when the catheter is to be withdrawn, initial withdrawal of the movement of the catheter, in the direction of arrow B in FIG. 8c, causes the catheter to move relative to the sleeve and the catheter to be drawn back into the sleeve 8 causing the electrodes 1 to be folded or bent down as shown in FIG. 8c, all to their starting position when the catheter is again fully home within the sleeve. In alternative embodiments the electrodes may be mounted to the mesh or electrode carrier of the catheter in another form, instead of by a spring mounting as described, by a pivot mount to the catheter. In the embodiment of FIG. 8 the spring 9 instead of a small coil spring may comprise a single resilient element of spring stainless steel or a resilient plastics material, for example.

For example an electrode array of a GI mapping catheter of the invention may comprise between 3 and 10 rows of electrodes spaced lengthwise of the catheter between the proximal end (coupled to tube 3) and the distal end, each row comprising between 3 and 10 electrodes spaced around the catheter, providing an array of between 9 and 100 electrodes for example. In an alternative embodiment the electrodes 1 may be arranged in rows angled or tangential to the longitudinal axis of the catheter, with, when the catheter is an expanding mesh catheter, an electrode at each or at least many intersections of mesh elements, over a part of the major surface area of the mesh catheter.

In relation to the electrode form, desired qualities for GI electrical signals acquired by the electrodes are an adequate signal to noise ratio (SNR) (the gastric mucosa has high impedance and attenuates signal), a stable baseline, and preferably a steep negative descent at the down-slope of the slow wave signal. As stated the electrodes are preferably protruding, to press into or indent the mucosa to achieve an adequate SNR. Smaller electrode diameters will generally achieve a steeper down-slope (shorter duration of activation over the electrode signal; quicker offset to onset period). However, if the electrodes are too protruding and of too small a diameter, they may puncture the gastric mucosa rather than press into it. A suitable form electrode may comprise a conductive protrusion of between 2 and 5 mm, or 2 and 3 mm, or about 2.5 mm in length (from the electrode carrier or electrode base to the tip of the electrode), and of a cross-sectional dimension (such as diameter if the electrodes have a circular or similar cross-section) of between 0.3 and 3 mm, or 0.5 and 1.5 mm, or 0.7 and 1 mm, or about 0.8 mm. The electrodes may suitably comprise sintered Ag—AgCl electrodes.

GI Mapping System and Method

In use a GI mapping catheter as described is connected by a cable to a signal acquisition stage of a GI electrical activity mapping system of the invention and once the GI catheter is positioned by the clinician in the GI tract, and engaged with the mucosal wall, the clinician may activate signal acquisition, typically via a graphical user interface. The GI mapping system is arranged to receive and process multi-channel electrical signals from the mapping catheter electrodes 1, either all or at least those making good contact, and is arranged to identify GI slow waves and spatially map the GI myenteric electrical activity (herein referred to as GI smooth muscle or slow wave electrical activity) preferably in real time or near-real time. The system may typically comprise a computer including a processor, program memory, and an operator interface including display or VDU which may be a touch-input screen and optionally also a keyboard or keypad, and a communications interface, coupled by a data bus.

The analysis processing by the GI mapping system of the electrical potential signals detected at the electrodes includes identifying GI electrical slow waves and mapping the electrical activity, which may include producing any one or more of an activation time map or maps of gastric electrical waves or wavefronts, a velocity field map or maps, an amplitude map or maps, all either as pixelated or isochronal maps or in other form, and which may also or alternatively animate any one or more of the same and/or GI slow wave propagation generally. The analysis processing may include mapping and/or animating the GI electrical activity or propagating waves over a generic or subject-specific anatomical model, running on the system processor.

The GI mapping system may also be arranged to carry out analysis processing including identifying any one or more of normal versus abnormal propagation or amplitudes, and dysrhythmias including focal activities, re-entrant loops, mechanisms of bradygastrias and tachygastrias and fibrillation for example. Thus analysis processing may also include comparing the mapped GI electrical activity to a stored reference database to provide an indication of normal or abnormal GI electrical activity.

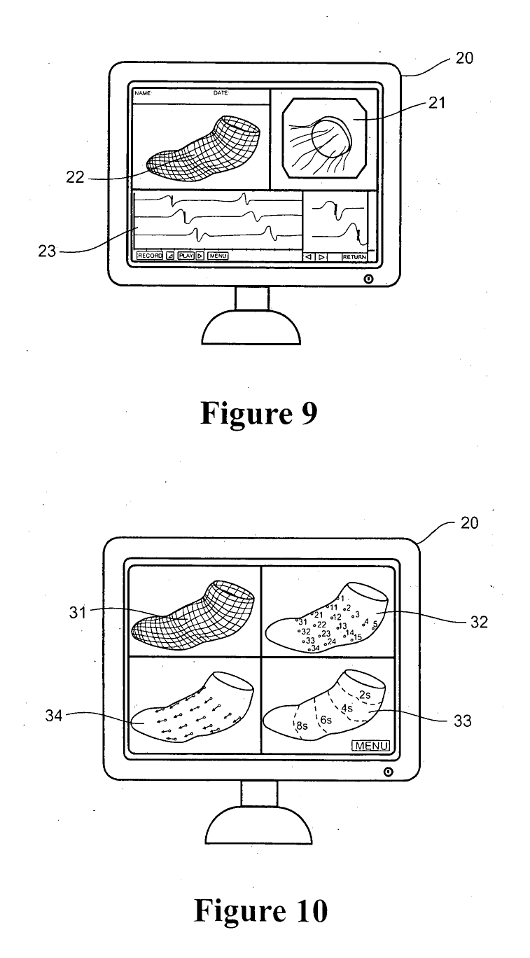

FIG. 9 shows an example of a user-display on a VDU 20 that a GI mapping system of the invention may present to a clinician during an examination. On the upper right indicated at 21 is a live video-endoscopy view of the gastrointestinal tract lumen. On the upper left indicated at 22 is a view of a generic or optionally subject-specific anatomical computer model of the section of the GI tract, over which the GI electrical activity or slow wave information obtained from the electrode array is mapped and may be animated. The live electrical potentials from a selection of channels from the electrode array are shown at 23. The system may be arranged to determine or approximate the relative locations of the electrodes in contact with the interior surface of the GI tract, to register same correctly to the model and optionally to develop or modify the model. The system may be arranged to display gastroscopic view 21 initially full screen, and after the mapping catheter is inserted and expanded the gastroscopic view may be reduced to the window 70 or closed, the electrophysiological recordings, and mapped electrophysiological data such as activation time map(s), velocity map(s), amplitude map(s), dysrhythmia map(s), and/or other wavefront propagation displayed as 2D or 3D images and/or animations shown in real-time. The system of the invention may also be arranged to record the session or to communicate the GI electrical data to another system for offline or further analysis and/or storage.

FIG. 10 shows another example of or an additionally available user display of a GI mapping system of the invention. A representation of an anatomical model of a stomach shape (or part thereof) is indicated at 31. The position of the electrodes of the array on the model (for example, for selecting channels to view) is indicated at 32. The electrode positions may be numbered. An activation time map which comprises isochronal propagation of GI slow waves on the stomach model is indicated at 33. An isochronal map comprises a two-dimensional contour plot showing the spatiotemporal sequence of GI slow wave activation. A velocity map which comprises multiple individual vectors on the model indicates the velocity and direction of GI slow wave propagation at each electrode is indicated on the model at 34.

The system may be arranged to produce and display and optionally animate on a model in 3D the GI electrical activity map(s).

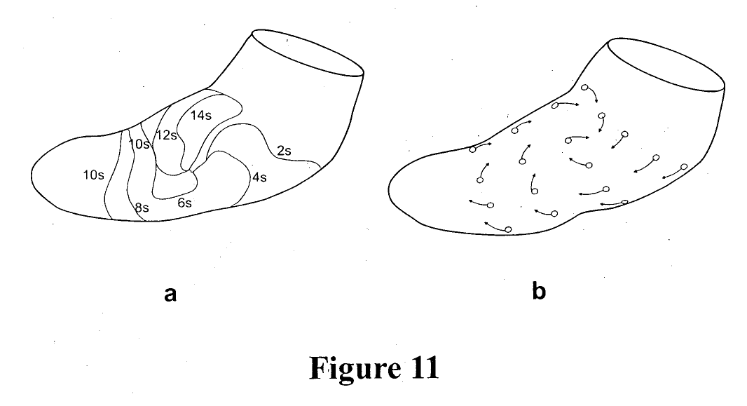

In FIG. 10, in the activation time map and velocity map at windows 33 and 34 the gastric electrical activity is shown propagating normally. FIGS. 11a and 11b show respectively similar activation time and velocity maps in which in contrast a GI slow wave is looping and propagating abnormally.

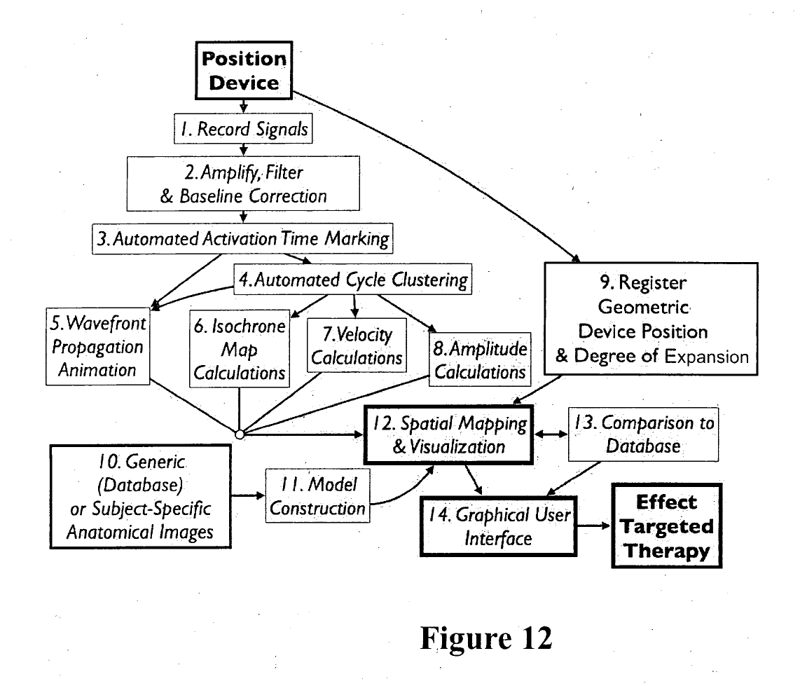

FIG. 12 is a flow chart illustrating signal analysis, mapping, and model fitting stages of a preferred embodiment of the invention. The darkest outline boxes indicate key user inputs, medium outline boxes indicate key integrated outputs, and lightest outline boxier indicate computer processing steps. After positioning a GI catheter and recording or beginning to record electrical signals from the electrodes, and any amplifying, filtering, and baseline correction, GI electrical slow wave events at electrodes are marked, and clustered or partitioned into clusters of electrical events each relating to a discrete GI electrical slow wave cycle.

One or more of velocity calculations, amplitude calculations, and isochrone map calculations are performed by the system processor. The resulting activation time, velocity, and amplitude information may then be spatially mapped in 2D or 3D in pixelated or isochronal or other form, optionally on a generic or subject-specific computer model of the GI tract or the part thereof. The model may be a stored generic model or one of a number of stored generic models of the GI tract or a part thereof, or may be constructed from a subject's specific anatomical images of the GI tract acquired prior to the EGG examination, for example via MRI or CT scanning. The catheter position and degree of expansion and thus individual electrode positions are registered on the map or model and the velocity, amplitude, and/or isochrone data fitted to the map or model, and displayed to the clinician on a VDU as 2D or 3D maps or animations. A wavefront propagation animation may be produced from the marked or marked and clustered GI slow wave events and also displayed. The system may be arranged to compare the mapped GI electrical activity to a database, and a clinician may interface with the system via a touch screen, keypad, computer mouse or similar through an appropriate menu or non-menu based interface system. The clinician may use the resulting analysis to effect targeted therapy for the patient.

Many of individual system blocks of the preferred embodiment system of FIG. 12 are now described in further detail.

Signal Recording

Signal acquisition may for example be at a sampling resolution of >1 Hz, typically at ˜30 Hz, and up to 512 Hz or greater. In a signal acquisition stage the signal channels may be digitized and amplified, and filtered to remove low frequency drift and wandering baselines, important for mucosally-acquired low amplitude and low frequency GI electrical signals, and to remove unwanted artifacts and noise.

Automated Activation Time Marking

“Activation” as used herein refers to a rhythmic spontaneous inward current in interstitial cells of Cajal, causing the cell membrane potential to rapidly rise. In extracellular recordings the onset of this depolarization termed “activation time” or AT signals the arrival of a propagating electrical wavefront to a particular location in the tissue. ATs must be identified (“marked”) at each electrode site. The marked electrode ATs are used to generate an activation time map or maps which provide(s) detailed spatiotemporal visualization of the spread of GI electrical activity across an area of tissue. ATs are identified to produce an activation time map or animation.

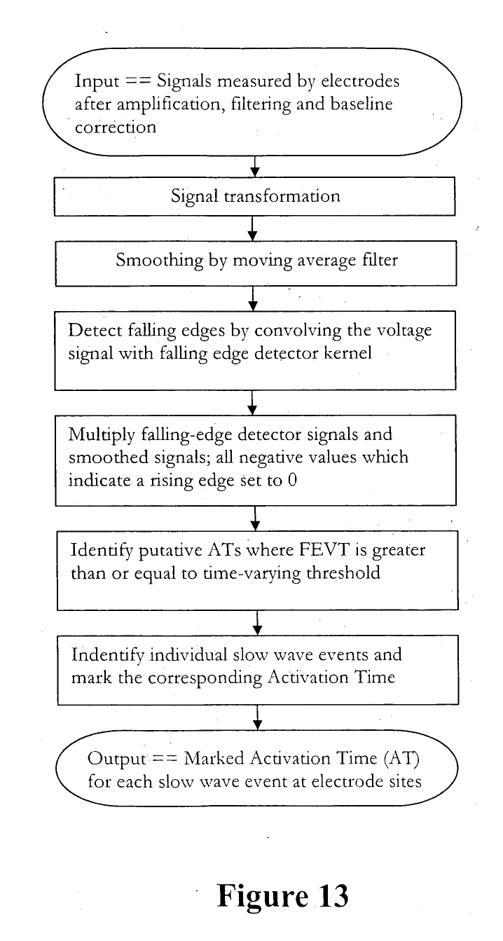

A preferred method for automated AT marking is a falling edge varying threshold method, which comprises transformation, smoothing, negative edge detection, time-varying threshold detection, and AT marking of the signal from each electrode. FIG. 13 is a flow chart of a preferred embodiment of an FEVT method for GI slow wave activation time identification.

Transformation can be carried out by for example negative derivative, amplitude sensitive differentiator transformation, non-linear energy operator transformation, or fourth-order differential energy operator transformation. A moving average filter of a tunable width is applied to the transformed signal to smooth the signal. The transformation amplifies the relatively large amplitude, high frequency components in the recorded signal, which corresponds to the onset of activation. Subsequent filtering increases the SNR of the transformation by reducing high frequency noise.

An edge detector kernel is then be used to identify falling edges within the smoothed signal. A falling edge produces a positive deflection in the signal from the edge detector kernel, and a rising edge produces a negative deflection.

A FEVT signal is then calculated by multiplying the signal from a falling-edge detector and the smoothed signal, and then all negative values which indicate a rising edge are set to 0.

In the preferred form a time-varying threshold is calculated from the FEVT output, by computing the median of the absolute deviation in a moving window of predefined width. The centre of the moving window consecutively shifts one sample forward, such that the threshold is computed for each point in time over the duration signal. Such a variable threshold improves detection accuracy by accounting for slight deviations in the waveforms of recorded signals. A constant threshold may be used but a time-varying threshold may reduce potential double counting and mis-marking. Signal values greater than or equal to the threshold define the times at which slow wave events might occur.

Individual slow wave events are then identified from the resulting data set which may contain multiple slow wave events, by imposing a criterion that distinct events must be separated by a minimum time.

Automated GI Slow Wave Cycle Clustering

The ATs as are clustered based on temporal closeness, into distinct cycles that partition the discrete propagating GI slow wave wavefronts. Clustering identifies individual GI slow waves based on a temporal closeness criterion, and proceeds in iterative fashion. Consecutive members in a data set are grouped as representing the same GI slow wave event if they are close enough in time to an estimated activation time. Such estimation employs deriving the best-fit second order polynomial surface, based on the location of electrode sites and the activation times detected at them. The estimated activation time is computed by extending said polynomial surface to the candidate location for clustering. The maximum time difference allowed to cluster two members is termed the time tolerance; its value must be long enough to accommodate small estimation errors and identify fractionated waveforms as single events, but short enough to properly partition distinct GI slow waves. When no more members of the data set meet this closeness criterion, a new cluster is formed to represent the next GI slow wave event. Auto clustering groups all marked data into individual clusters, each delimiting an independent GI slow wave event

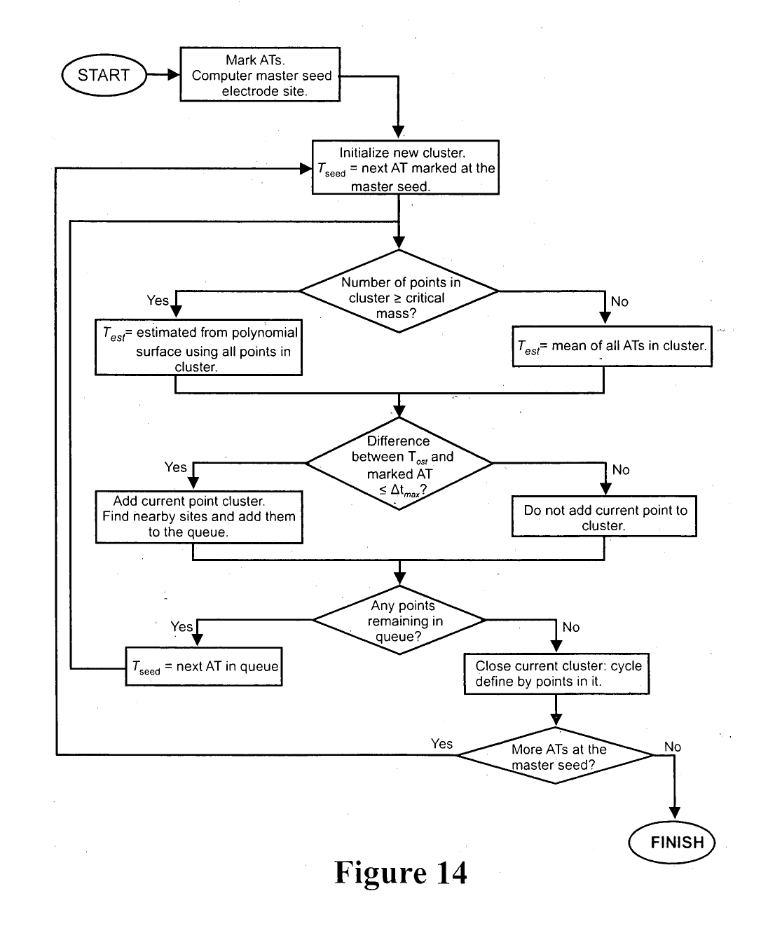

FIG. 14 is a flow chart of a preferred embodiment clustering method termed region growing using polynomial surface estimate stabilization (REGROUPS) for clustering (x, y, t) points representing ATs into groups representing independent GI slow wave cycles, where (x, y) denotes the position of an electrode site and t denotes an AT marked at that site, and t denotes the activation times identified at that site.

The algorithm is initialized by automatically selecting a “master seed”, which is an electrode position embedded in a region with the maximal density of information about a propagating wavefront. The cluster is then grown outward from the region where the spatial density of data is highest, ensuring that the subset of points initially assigned to the cluster is statistically cohesive and limiting the possibilities of assigning noise signals to a nascent clusters. The master seed may be selected by first calculating the total number of ATs detected at each electrode site, then finding the centre of mass and selecting the seed location as the electrode closest to the centre of mass. Once the master seed is located, a queue containing the nearby electrode sites' ATs in a specified circular range of the master seed is created and the first AT in the queue becomes the current seed. Each AT is tested for membership of a cluster based on comparison to an estimated AT, which is derived by fitting (in the least squares sense) a second-order polynomial surface to the data points already assigned to the cluster. The 2^{nd} order surface acts as a continuously updating spatiotemporal filter: if the time difference of estimated AT and tested AT is small enough, then the tested AT is considered as representing a same wavefront as the seed and is assigned to the cluster. Once assigned, the point is not assessed again. If the tested point is clustered, all of its neighbour electrodes and marked ATs at these electrodes are added to the back of the queue, providing they are not already in it. If a tested point is not clustered, it may be tested again for membership only after a new cluster is initialized at the next iteration. This restriction forces all wavefronts to be independent. Regardless of whether any point is added to the cluster, the current seed is removed from the queue and the next electrode site becomes the current seed. Thus, the region in (x, y, t) space representing an independent cycle grows, and terminates when the queue of nearby points becomes empty. At this stage, the cluster contains all ATs from one GI slow wave cycle. The same process is repeated to identify another independent cycle, starting with the next sequential AT marked at the master seed. Each iteration produces a cluster of (x, y, t) points which represent the dynamics of an independent GI slow wave cycle, from which wave front propagation, an actuation time map may be produced, and isochrones map calculation, velocity and amplitude calculation can all be realized.

Activation Time or Isochronal Mapping

An activation time or isochronal map comprises a contour plot of GI slow wave activation. An isochronal map may comprise a spatial representation of the electrode sites, and the isochrones (contour lines), which represent the spatial distribution of ATs lying within the same specified time window, i.e. sites with similar activation times. In a preferred form the temporal resolution (i.e. isochrone interval) may be about 0.5 seconds when the activity is fast (>10 mm/s), about 2 seconds when the activity is slow (<4 mm/s), and about 1 second when the activity is from 4-10 mm/s, for example. Information such as speed and direction of propagation may be inferred from an isochronal map.

The spatial interval of two neighboring isochrones can be used to calculate the velocity of slow wave propagation.

An activation time or isochronal map may be produced by:
Plotting the identified ATs in the same spatial arrangement as the electrodes.
Mapping the ATs to the electrodes to which they pertain, in the same configuration as the electrode matrix. The value of each AT may be represented by a colour or colour tone in a colour or colour tone spectrum that represents the appropriate range for the activation values. A look up ‘configuration file’ may contain information on electrode distribution and inter-electrode distance; the electrode numbers may be stored in a matrix, with the corresponding electrode number reference by the indices.

A pixelated isochronal map may be converted into a smooth, filled contour map with isochronal lines spaced at a specified time interval.

Poor electrode contact to the mucosal surface may result in areas with imperfect electrical recordings. To represent the entire activation field, areas with bad contact may be interpolated based on the surrounding ATs. Inactive electrode sites surrounded by several active sites are preferably interpolated into the AT map. In a preferred form a 2-stage spatial interpolation and visualization scheme may conservatively interpolate inactive electrodes using information from neighboring active electrodes on the basis that if an inactive electrode site is bordered by three directly adjacent (including diagonal) active electrodes, the AT is linearly interpolated from adjacent active sites' ATs, and correspondingly pseudo-colored (an “interpolated site”). If the total number of active plus interpolated sites bordering a still-blank site is four, then the still-blank site in interpolated. Such a 2-stage scheme, as opposed to a recursive one, prevents a run-away interpolation process from inappropriately filling in blank sites across the entire array.

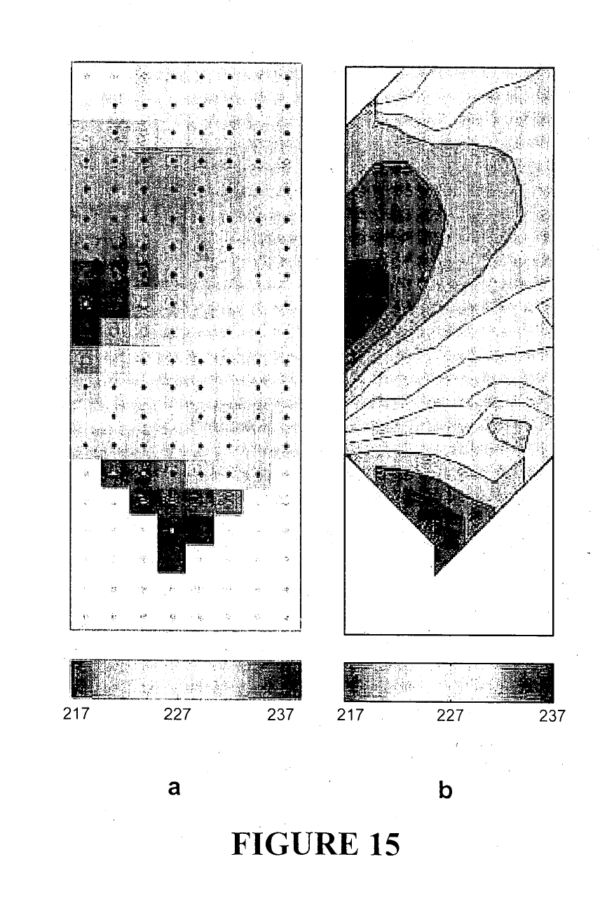

FIG. 15a shows a pixelated isochronal map or a part thereof and FIG. 15b shows such a smooth filled contour map with isochronal lines. In FIG. 15a black dots indicate electrode sites at which an AT was marked, and white dots indicate electrode sites for which no AT was marked, but in some cases was interpolated. The ATs are color coded to propagate from for example red to blue, representing the earliest and latest ATs respectively over a 20 second interval from second 217 to second 237. In FIG. 15b the isochronal lines are spaced at 2 second intervals.

An isochronal map may also be applied over an anatomical geometry model in 2D or 3D to aid visualization and accurate diagnosis for the clinician.

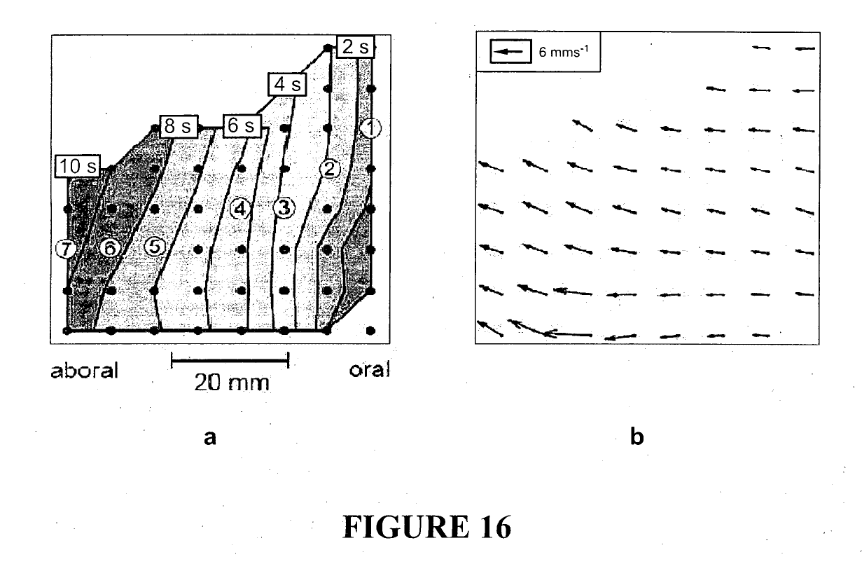

A velocity field may be mapped in 2D or displayed over anatomical organ geometry in 3D in a similar way to as described for activation time mapping. FIG. 16a is an isochronal activation time map, and FIG. 16d is a calculated velocity field map . . .

Wavefront Propagation Animation

The wavefront propagation may be directly animated from the ATs, or clustered ATs to provide animations of an improved accuracy or clearer visualization to convey information of a propagation wave behaviour, including complex behaviors such as occur in slow wave dysrhythmias. Separate colors may be assigned to the discrete wavefronts in the animations (or map(s)). In one embodiment, animation may be performed by:
Configuring a computational array in the same configuration as the recording electrodes array.
Checking each location on the recording electrode array at each specified time frame (for example at 1 second intervals), and if an AT occurred at that electrode within that time frame, then representing the pointer pixel in the computational array corresponding to the location of the electrode highlighted or in a different colour than those electrodes at rest.
Causing the thus ‘activated’ electrode(s) to stay highlighted or coloured for a fixed duration before turning off again (i.e. going back to ‘rest’). The highlighted or coloured point may fade as it disappears.
Different colours may be assigned to distinct clusters each relating to a discrete GI electrical wave, for example based on a repeating pattern of a few colours.

Animation(s) may also be on an anatomical geometry model to aid visualization and accurate diagnosis for the clinician as will be further described. Preferably the animation(s) may be zoomed and rotated.

Velocity Calculations and Mapping

GI slow wave propagation velocity in the stomach varies. Differences may be greater during dysrhythmia. Velocity calculations may assist in diagnosing at least some dysrhythmias.

A preferred velocity calculation method comprises a fitting and calculation process. To calculate a uniform spatially-distributed velocity field, the ATs from each GI slow wave are first interpolated, for example using the following second-order polynomial:

T(x,y)={p(1), p(2), p(3), p(4), p(5), p(6)}·{x̂2, ŷ2, xy, x, y}

where T(x,y) is the interpolated activation times at location x and y in the electrode array. The array of p contains six coefficients for the second-order polynomial. The AT events in an isochrone map is fitted in a least-square sense using the following formula:

Ap={t(1) . . . t(n)}={x(1)̂2, y(1)̂2, x(1)y(1), x(1), y(1); . . . ; x(n)̂2, y(n)̂2, x(n)y(n), x(n), y(n)}·{p(1), p(2), p(3), p(4), p(5), p(6)}

where t is the automatically identified activation times of slow wave events. Matrix $A$ contains evaluated terms using the x and y coordinates of the corresponding activation time. For solution for p is solved by using the singular value decomposition of A into V, S, and U such that,

A=VSÛT.

The search parameters for the number of events included in one wave are applied over the entire set of electrodes within the isochrone map. For the description of normal events, the number of active electrodes within for example a 16×16 array may be adequately fitted by a second-order polynomial due to the slow moving wave front of the gastric slow waves.

Velocity is calculated using the following equation:

V(x,y)={dx/dt; dy/dt}={Tx̂2/(Tx̂2+Tŷ2); Tŷ2/(Tx̂2+Tŷ2)}, where

Tx=dT(x,y)/dx and Ty=dT(x,y)/dy.

This velocity calculation procedure ensures that the velocity vector is calculated orthogonal to the wavefront, i.e. representing the true direction of propagation.

Less preferably velocity may be calculated via a finite-difference based derivative estimation from neighbouring electrodes.

Amplitude Calculation and Mapping

Extracellularly-recorded slow wave amplitudes may be indicative of pathology and/or dysrhythmia because amplitudes may be low in some diseases, where interstitial cell of Cajal networks are degraded and/or dysrhythmia may be associated with regional high or low slow wave amplitudes.

A slow wave amplitude may be calculated based on the identified AT of an event.

A preferred amplitude calculation algorithm to calculate amplitude from the processed signals is:

A=|max[S(t(i)−1.5)−S(t(i)+6)]−min[S(t(i)−1.5)−S(t(i)+6)]|

where S(i) denotes the processed a slow wave signal in a channel at AT of i. The amplitude is the absolute difference between the maximum and the minimum in the signal 1.5 seconds, for example, before to 6 seconds, for example, after the identified TA. This interval captures the entire duration of the depolarization (down-stroke) the repolarization (return to baseline) of a gastric slow wave event, while still within the time interval of a single slow wave event, i.e. unlikely to run into the signal of the next slow wave event due to the refractory period being longer than 6 seconds.

Registration of Device Position and Expansion

The electrode array position may be anatomically registered in the GI tract by for example:
The system maybe arranged to display the position of the mapping catheter in a model stomach geometry which in conjunction with a displayed an endoscopic view assists the clinician to position the catheter where desired.
By a second roving anatomical catheter arranged to a low-current locator signal to a reference electrode, measuring and transmitting samples, against a 3D referencing system, for the construction of a geometric matrix or ‘virtual lumen’. The position of the mapping catheter and electrode array is also registered within this matrix by the 2^{nd} catheter.
By imaging e.g., plain film radiography in 2 axes, and then forming a mesh based on the identified electrode positions.

In one embodiment a measuring system is arranged to measure the volume of air or other fluid installed into an inflatable mapping catheter via a syringe or pump. The user instills a sufficient volume until the electrodes press against the gastrointestinal tract mucosa. Air may also be removed from the tract, via endoscopic suction, such that the tract walls collapse down around the device. The degree of inflation determines the final spacing of the electrode array because the electrodes move further apart during inflation. In a preferred embodiment the electrode spacing at the time of mapping is determined by:
The value of air of liquid instilled is measured, for example visually identified by a volume scale on the syringe or other device used to effect the inflation.
This volume is input by the user into the system.
The post-inflation surface area of the device is calculated by the system.
The spacing of the electrodes at the time of mapping is calculated by geometric calculations that define the distance between points on a 3-dimensional surface, with these distances being proportional to the degree of inflation.

The calculated ‘inter-electrode distance’ on the expanded device, at the time of mapping, is subsequently used by the system in calculating the activation times, clustering, isochrone, velocity, and amplitude mapping and animations.

Model Selection from Generic Database, or Subject-Specific Model Development

A subject-specific anatomical model of the mapped part of the GI tract may be produced by for example:
A medical image or image set providing a 2D or 3D description of an organ position is obtained, for example via ultrasound, MRI, CT, or plain abdominal x-ray of the patient.
The GI tract section of interest is extracted via manual (tracing the organ outline) or automated (determined by imaging density transition zones) segmentation methods to create a 3D data cloud representing the surface of the GI tract section.
A finite element mesh is created to match these data points using a non-linear iterative fitting method.

The system may comprise a database of multiple models along with corresponding data on how each was acquired e.g. sex, age, imaging methodology, medical history, pathological conditions, and an appropriate model may be recalled from the database by the system based on data such as demographic data relating to the patient entered by the clinician, for example the patients' sex and age data. For example, if a 5 year old female child is being examined, a mean stomach geometry for five-year old female children can be automatically presented to the clinician. Alternatively, a library of models may be stored for review by the clinician, to manually select one that best matching the stomach geometry of the patient under examination. This library is arranged in size order for intuitive browsing.

Model Construction and Mapping to Model

Construction of a specific anatomical model brings together:
registration of the mapping catheter position and degree of expansion, and
the anatomical stomach geometry model chosen by the clinician

to create a model specific for the GI tract section and patient under evaluation. The chosen anatomical geometry model is reconfigured to match the calculated geometry resulting from the mapping catheter expansion, for example by:
The calculated geometry of the expanded electrode array geometry is used as the ‘true’ reference geometry, being empirically determined at the time of the procedure.
The reference model geometry is resized by geometrically expanding or reducing the model proportions until they match the ‘true’ reference geometry proportions at the position of the mapping catheter within the GI tract.

With a specific model that best represents the anatomy under evaluation, and the position and degree of expansion of the mapping catheter, and electrode array, 2D or 3D activation time, velocity, and amplitude maps and animations may be applied to the model and displayed as referred to previously. For example this may be achieved by:
Common landmark points on the model and the locations of the recordings relative to these landmark points are identified in the model.
The root mean squared distances between these common points are minimized.
Activation time, velocity and amplitude maps are “texture mapped” or orthogonally projected onto the surface of the model.
Results from multiple recording sites can be combined to enable results from different regions to be compared in the relative locations at which they were recorded.

Analysis Comparison to Database

The system and method of the invention may facilitate an accurate diagnosis by allowing the clinician to compare the mapped GI slow wave data to standard reference (normal population) data. The system may be arranged to alert the clinician that the mapped characteristics deviate from the normal range in one or more ways. A specific diagnosis may be automatically suggested by the system, based on characteristic differences from the normal population.

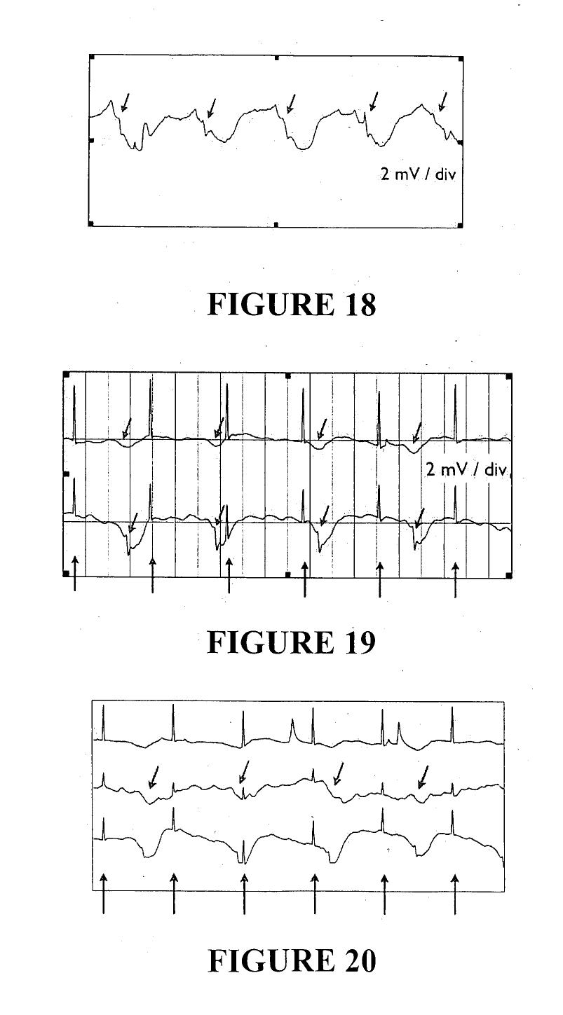

For example to detect low amplitude slow wave activity (low slow wave amplitudes may theoretically occur in gastroparesis due to degradation of the interstitial cell of Cajal networks), activation times in individual slow wave cycles may be identified and amplitudes calculated. In a user menu in the system interface, the clinician may select to review slow wave amplitudes for a specific time period of the recording. As well as spatially mapping the amplitudes for the selected time period, the system is arranged to perform the following steps to present a comparison to the standard reference range:
Average the amplitudes across every slow wave i.e., calculation of a mean and standard deviation for each cycle.
Average amplitudes across all waves to generate a mean and standard error of the mean.
Statistically compare the resultant values to a standard reference database of normal data obtained from a control population without gastric pathology (see FIG. 18a below).
Display the result. For example, if the slow wave amplitudes of the patient with gastroparesis are statistically found to be lower than that of the standard reference range, a display item will state this fact. The clinician may note the finding, and conclude that reduced slow wave amplitudes are a marker of poor stomach contractility, contributing to a diagnosis.

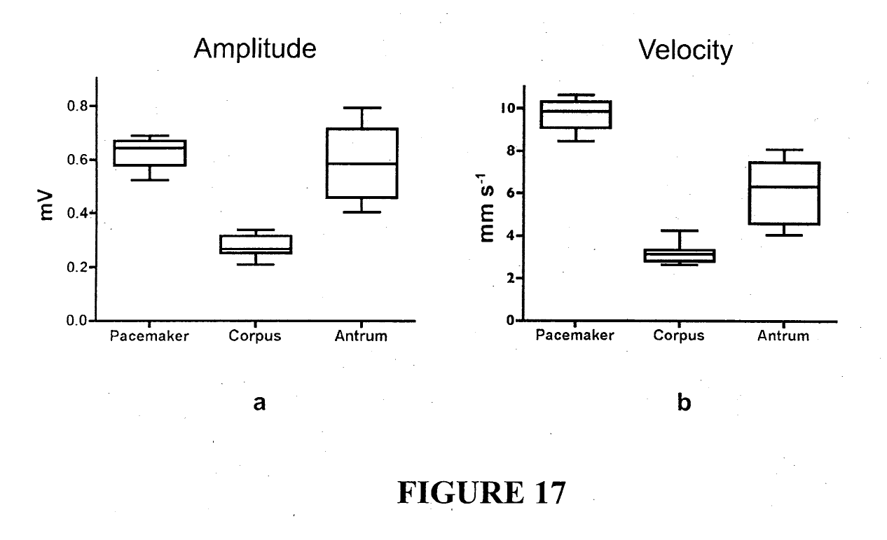

FIGS. 17a and 17b show standard reference ranges (normal human population) of slow wave amplitudes and velocities respectively in different gastric regions. Note these are serosal reference data, mucosal data will has lower amplitudes due to signal attenuation by a mucosa, and a calibration factor must be applied.

As a further example, to detect dysrhythmic slow wave propagation (anisotropic slow wave propagation and re-entrant circuits may occur during dysrhythmia), activation times of individual slow wave cycles are identified and isochronal activation maps and velocity maps are calculated for every wave cycle. In a user menu in the software interface, the clinician may select to review slow wave propagation and velocity for a specific time period of the recording i.e. specific slow wave cycles occurring during that period. As well as spatially, mapping the isochronal activation patterns and velocities for the selected time period, the system is arranged to perform the following steps to present a comparison to the standard reference range:
Average the velocities of each cycle to calculate a statistical mean velocity and standard deviation for each cycle, and preferably separate the longitudinal and circumferential velocity components.
Average velocities across all cycles are calculated to generate a mean and standard error of the mean for the total velocity, and the total longitudinal and circumferential velocities.
The resultant values are statistically compared to a standard reference database of normal data obtained from a control population without gastric pathology (see FIG. 18b).
The result is displayed in the software interface. For example, if the circumferential components of the slow wave velocities of a patient with functional dyspepsia are statistically found to be higher than that of the standard reference range (i.e. ˜zero mm/s circumferential propagation in the normal human antrum, then a display item indicates this. The clinician may note the finding, and conclude that an antral dysrhythmia is occurring, contributing to a diagnosis.

The clinician may then institute a targeted therapy into the location where the dysrhythmia is occurring, such as pharmaceutical agent, or pacing or ablation therapy, to interrupt the dysrhythmic mechanism. The targeting of this therapy can be specifically guided by the anatomically visualized spatially represented isochronal slow wave maps, or animations, to ensure it is accurately delivered.

Gastric Stimulation or Pacing and Entrainment Mapping

The GI mapping catheter and system may also be used to deliver targeted stimulation therapy through at least some electrodes for diagnostic or therapeutic purposes. The stimulation dose and its effects on GI electrical activity may be measured via the rest of the electrode array. It may be used in this way to guide stimulation lead implantation, or for other treatments such as targeted electrical pathway ablation or drug delivery, for example. The GI electrical activity mapping system and method of the invention may be used for mapping GI electrical activity changes resulting from gastric pacing (referred to herein as entrainment mapping). Gastric stimulation involves delivery of electrical current into the myenteric layers of the stomach to induce beneficial effects on nerve function, electrical activity or symptoms. Gastric pacing involves electrically stimulating the stomach specifically in order to mediate (entrain) the propagation of GI slow waves for therapeutic purposes. Gastric stimulation and pacing have primarily been researched for the treatment of gastroparesis and obesity. In gastroparesis, gastric pacing may revert gastric dysrhythmias, normalise motility and emptying, and thereby control symptoms. In obesity, gastric pacing may controllably disrupt or reverse normal GI slow wave activity, with the aim of restricting eating and inducing satiety.

Entrainment mapping allows an accurate spatiotemporal evaluation of pacing outcomes. The interaction between the native and entrained activities can be defined by entrainment mapping, dysrhythmias can be accurately observed, and the area of tissue affected by a pacing protocol can be quantified across the mapped area. The velocity of slow wave propagation in all directions can be determined by entrainment mapping. The changes in amplitude can also be determined by entrainment mapping.

Entrainment mapping may be employed when applying gastric pacing via multiple coordinated electrode sites (‘multi-channel stimulation’) to improve the efficacy and energy-efficiency of gastric pacing. Entrainment mapping may be used to study slow wave behaviours because it enables an accurate and detailed analysis of multiple local slow wave events surrounding each stimulus point, and their subsequent interactions.

### EXAMPLES

The invention is further illustrated, by way of example and without intending to be limiting, by the following description of trials work.

### Example 1

### Trial

Method

A mapping catheter was constructed from: a 24 Fr two-way urinary catheter (outer catheter), a 12 Fr nasogastric tube (inner catheter), a latex balloon (standard condom), 32 ECG-dot central pins (stainless steel contact surfaces), 32 copper wire leads (connected to a 68-way SCSI ribbon cable; soldered at each end), and a three-way tap and inflation syringe. The catheters were joined with heat-shrink tubing, and the ECG dots were stuck to the balloon with glue.

The configuration of the balloon electrode array was circumferential and was numbered as follows (proximal to distal):

| | | | | | | | |
|---|---|---|---|---|---|---|---|
| 7 | 9 | 23 | 24 | 29 | 30 | 17 | 26 |
| 31 | 15 | 12 | 32 | 10 | 3 | 5 | 27 |
| 28 | 13 | 11 | 14 | 6 | 8 | 4 | 25 |
| 16 | 1 | 2 | 20 | 18 | 22 | 21 | 19 |

The SCSI cable connector pins were connected to port A of the ActiveTwo System (BioSemi, Netherlands). A flexible printed circuit board mounting a number of electrodes was connected to port B, to allow validation against a serosal reference electrode.

A female weaner cross-breed pig of 39 kg was fasted overnight and anaesthetised. A small midline laparotomy incision was performed and the prototype device was placed on the serosal surface, for a recording duration of 5 minutes. The distal stomach was then brought into the wound and a mini gastric stoma was fashioned. A gastric stoma was used for insertion of an array of electrodes to contact the mucosa on the interior of the gastric wall instead of endoscopic access because endoscopic access is very difficult in the pig due to its restrictive anatomical configuration in the posterior oropharynx, and a mini-laparotomy was necessary in any case to perform simultaneous reference electrode mapping. The PCB carrying reference electrodes was placed over one row of the mucosal electrodes (palpable through the gastric wall) and a 10 minute recording was taken.

Unipolar recordings were acquired from the devices at a recording frequency of 512 Hz. Each device was connected to the ActiveTwo via a 1.5 m 68-way ribbon cable, which was in turn fibre-optically connected to a notebook computer. Signals from all electrode channels were filtered using a second-order Bessel low-pass filter of 10 Hz.

The activation times of the slow wave events were marked at the point of maximum negative slope. The normalized activation times were plotted in the same spatial arrangement as the endoscopic prototype and PCB electrodes. Interpolation of electrodes that had not adequately recorded the slow wave activation was performed using the linear interpolation scheme that is programmed in the ‘linear’ method in the grid data function in Matlab. Three further iterations of uniform linear interpolations were performed on the activation times to smooth the isochrones of activation times.

Isochrones were then calculated from the activation times at 1 or 2 second intervals, showing the timing and direction of slow wave propagation. In order to calculate a uniform spatially-distributed velocity field, the activation times from each wave were first interpolated using the following second-order polynomial:

T(x,y)=p(1)x²+p(2)y²+p(3)xy+p(4)x+p(5)y+p(6)

where T(x,y) is the interpolated activation times at location x and y in the electrode array. The array of p contains six coefficients for the second-order polynomial. A least-square-fitting algorithm was used to calculate the polynomial coefficients:

$$Ap = [ \begin{matrix}{t}_{1} \\ ⋮ \\ ⋮ \\ ⋮ \\ ⋮ \\ {t}_{n}\end{matrix} ] = [ \begin{matrix}{x}_{1}^{2} & {y}_{1}^{2} & {x}_{1} {y}_{1} & {x}_{1} & {y}_{1} & 1 \\ ⋮ & ⋮ & ⋮ & ⋮ & ⋮ & ⋮ \\ ⋮ & ⋮ & ⋮ & ⋮ & ⋮ & ⋮ \\ ⋮ & ⋮ & ⋮ & ⋮ & ⋮ & ⋮ \\ ⋮ & ⋮ & ⋮ & ⋮ & ⋮ & ⋮ \\ {x}_{n}^{2} & {y}_{n}^{2} & {x}_{n} {y}_{n} & {x}_{n} & {y}_{n} & 1\end{matrix} ] [ \begin{matrix}{p}_{1} \\ {p}_{2} \\ {p}_{3} \\ {p}_{4} \\ {p}_{5} \\ {p}_{6}\end{matrix} ]$$

The polynomial coefficient (p) was solved by

p=VS⁻¹U^{T}t

where t is the automatically identified activation times of slow wave events. The above matrix contains evaluated terms using the x and y coordinates of the corresponding activation time. The solution for p was solved by using the singular value decomposition of A into V, S, and U (A=V SUT). The search parameters for the number of events included in one wave were over the entire set of electrodes (Δx=99 mm; Δy=27 mm) within a 10 s interval (Δt=10 s). For the description of normal events, the number of active electrodes within the array was adequately fitted by a second-order polynomial due to the slow moving wavefront of the gastric slow waves. Velocity was calculated using the following equation:

$$V ( x , y ) = [ \begin{matrix}\frac{\partial x}{\partial t} \\ \frac{\partial x}{\partial t}\end{matrix} ] = [ \begin{matrix}\frac{{T}_{x}}{{T}_{x}^{2} + {T}_{y}^{2}} \\ \frac{{T}_{y}}{{T}_{x}^{2} + {T}_{y}^{2}}\end{matrix} ] , \\ {T}_{x} = \frac{\partial T}{\partial x} , \\ {T}_{y} = \frac{\partial T}{\partial y}$$

where V(x;y) is the velocity vector evaluated at coordinates x and y on the electrode array.

Slow wave amplitudes were calculated. Where appropriate, slow wave parameters were averaged over multiple successive waves and expressed as means±s.d., and Students' t-test was used to evaluate for statistical significance.

Subsequently, recording channels 1 and 2 of the catheter were disconnected from the BioSemi, and reconnected to a stimulator (World Precision Instruments, Saresota, Fla.), and a continuous bipolar stimulation protocol of amplitude 3 mA, pulse width period 300 ms and period 17 s was delivered.

Results

FIG. 18 shows a slow wave data recorded from the serosal surface of the GI tract. The mean serosal slow wave amplitude recorded by the prototype was 0.20+/−0.06 mV.

Slow waves were recorded in a number of channels from the catheter electrodes. FIG. 19 shows slow wave activity and stimulation artifact recordings taken from the mucosal gastric surface (window=100 s). The top channel is from an electrode of the mapping catheter and the bottom channel is from the adjacent PCB reference. The regular sharp peaks indicated by the upwardly pointing arrows show stimulation artifacts. The downwardly pointing arrows indicate the slow waves. Evaluation of the slow wave data confirmed that there was a precise 1:1 coupling of the interval period between the mapping catheter electrodes and the reference electrodes. Similarly, the frequency of slow wave events at the mapping electrodes was the same as the frequency of events in the reference electrodes. FIG. 20 shows recordings from two adjacent catheter electrode channels, showing certain slow wave events.

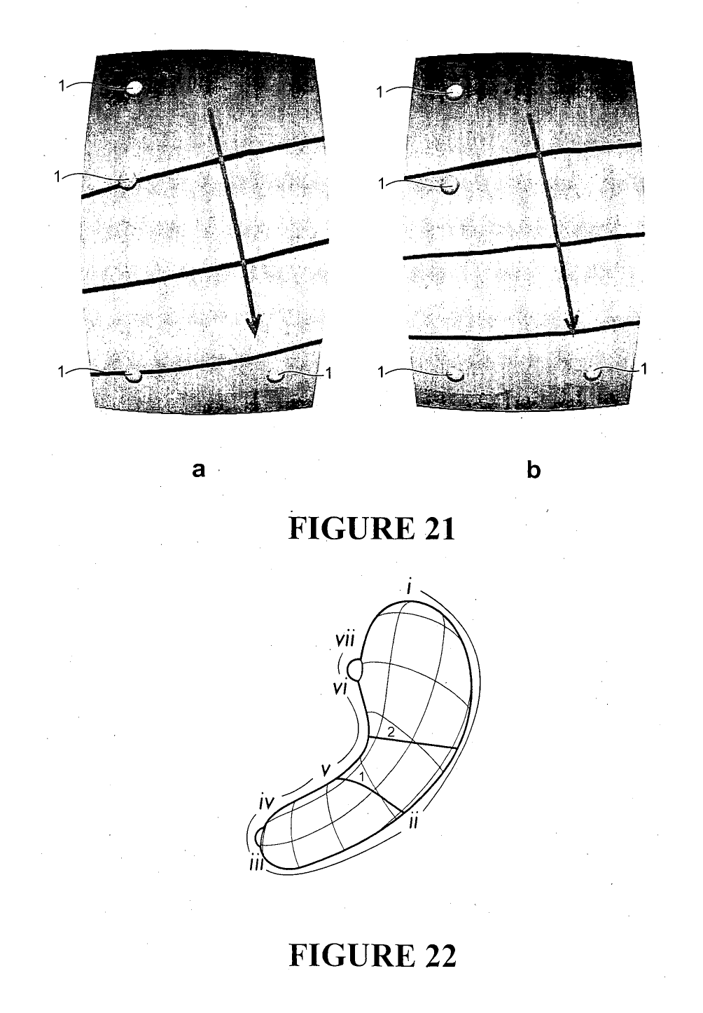

FIGS. 21a and b show spatial activation maps from two consecutive waves, demonstrating normal aboral slow wave propagation and computed velocities of 0.34 cm s⁻¹—FIG. 21a, and 0.31 cm s⁻¹—FIG. 21b, being consistent with the velocity field measurements calculated from the serosal refence electrodes (0.39+/−0.06 cm s⁻¹). These were generated by linear interpolation over the represented electrodes indicated at 1, according to the array dimensions measured from the inflated balloon. The dark transverse lines indicate slow wave propagation and the top-to-bottom arrows the direction of propagation.

In summary the system was successfully able to register slow wave activity from the mucosal surface, verified as true slow wave activity against the reference electrodes, recording simultaneously on the serosal surface. Spatial activation maps were generated from the mucosally-recorded data demonstrating the local propagation frequency, direction, activation pattern and velocity.

### Example 2

### Trial

Method

Flexible PCB multi-electrode recording arrays consisting of copper wires and gold contacts on a polyimide ribbon base were employed. The recording head of each array had 32 electrodes in a 4×8 array, at an interelectrode distance of 7.6 mm.

Mapping was undertaken in human subjects undergoing upper abdominal surgery, immediately after laparotomy and prior to additional surgical dissection. Up to 6 PCBs (192 electrodes; ˜93 cm²) were used in each experiment, and were held together in ideal parallel alignment. The recording surface of the PCBs were positioned flush with the anterior serosal surface of the stomach. The posterior gastric surface was not mapped. The recording period was 10-15 minutes, usually allowing two adjacent areas of gastric tissue to be mapped.

Unipolar recordings of 10-15 min duration were acquired using the ActiveTwo System (Biosemi, Amsterdam), at a recording frequency of 512 Hz. The common sense (CMS) and right leg drive (DRL) electrodes were placed on the right upper torso of each patient. Each PCB was connected to the ActiveTwo in turn connected to a notebook computer. Signals from all channels were filtered using a second-order Bessel low-pass filter of 10 Hz. Following each experiment, the activation times of the slow wave events were marked at the point of maximum negative slope. Activation maps depicting propagation sequences were computed by interpolating the activation times over the recorded area and using triangulation techniques to compute isochronal bands. Slow wave velocities were computed by taking the gradient of the isochronal fields as described in Example 1 above and slow wave amplitudes were calculated as described in Example 1 above. Where appropriate, slow wave parameters were averaged over multiple successive waves and expressed as means and SEM, and an ANOVA, Students' T-test, or a linear mixed model with a random term for intercept and site was used to test for statistical significance depending on the variables that were being compared. The pacemaker region was defined as the area covered during the first two seconds of slow wave propagation for the purposes of these statistical correlations.

The table below gives slow wave amplitudes and velocities across the gastric regions for 11 patients, (means±sem). Adjacent gastric regions showed significant differences (p<0.05).

| | Pacemaker | Corpus | Antrum |
|---|---|---|---|
| Amplitude | 0.62 ± 0.04 | 0.27 ± 0.02 | 0.58 ± 0.05 |
| Velocity | 0.97 ± 0.05 | 0.32 ± 0.02 | 0.61 ± 0.07 |

Anatomical Registration and 3D Visualisation

The geometry of the stomach of patients was used to create a subject-specific anatomical mesh, upon which the relevant physiological data was registered. To develop each patient's mesh, a pre-operative computed tomography (CT) scan was retrieved and the stomach outline was digitised on each two-dimensional axial image to form a subject specific stomach model. The digitised points from each image were then registered in 3D space to create a cloud of points representing the outline of the stomach surface. A bicubic Hermite finite element mesh was then used to represent these digitised points by minimising the orthogonal projections between each data point and the surface of the mesh.

The stomach is distensible and its surface dimensions and volume are dependent on the quantity of contained solids, liquids and gases. Mapping was performed in the intra-operative state, when the stomach was empty of solids and liquids and was relatively collapsed. Therefore, in order to achieve accurate anatomical registration, multiple intra-operative measurements of the stomach surface were obtained between fixed anatomical points at the time of mapping, along both the greater and lesser curvatures and across the transverse organ axis. The specific anatomical points used were: the apex of the fundus, the boundaries of the gastroesophageal junction and the pylorus, and the point of the angularis incisura and its opposite point on the greater curvature located at approximately 45° from the angularis. Each stomach was measured during surgery, between points i-vii, and across lines 1 and 2, as indicated in FIG. 22. These measurements were used to reconfigure the subject-specific stomach models so that the recorded physiological data could be accurately registered. The size of each subject-specific mesh generated from the pre-operative CT scan was then adjusted to match these intra-operative measurements for each patient.

For each patient, the PCB placements and physiological data (time activation maps and slow wave velocity field maps) were registered on the 3D subject-specific model. This was achieved by using a non-linear search to minimise the distances between at least three common key landmark points determined during the study (e.g., distances between an electrode and specified locations on the stomach). The physiological data was then orthogonally projected onto the surface of the stomach model.

Results

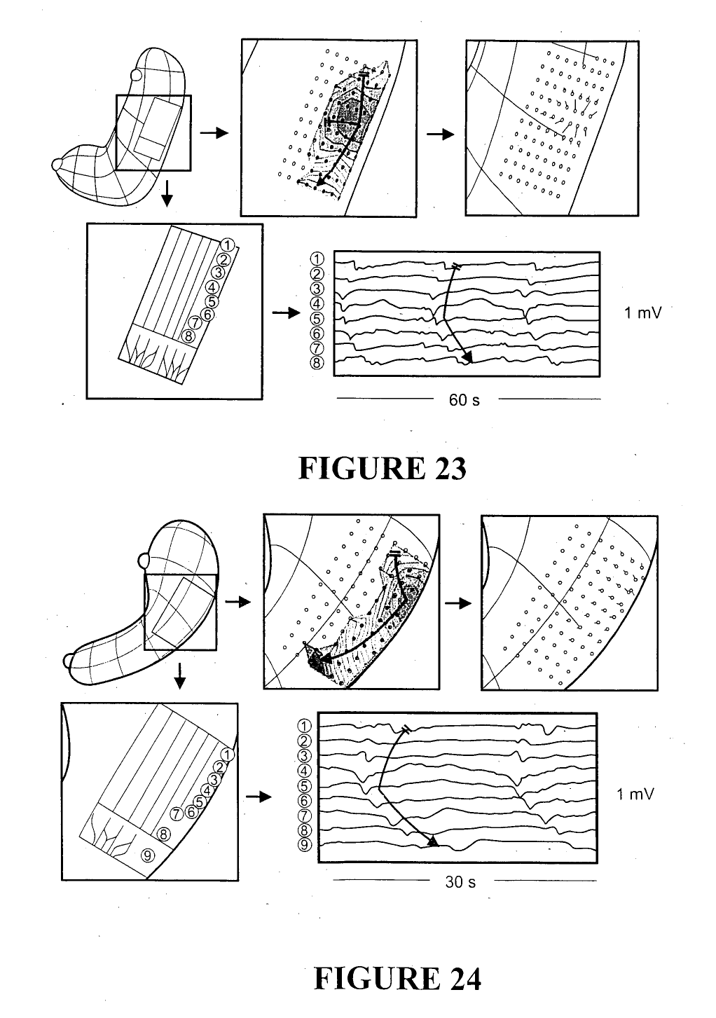

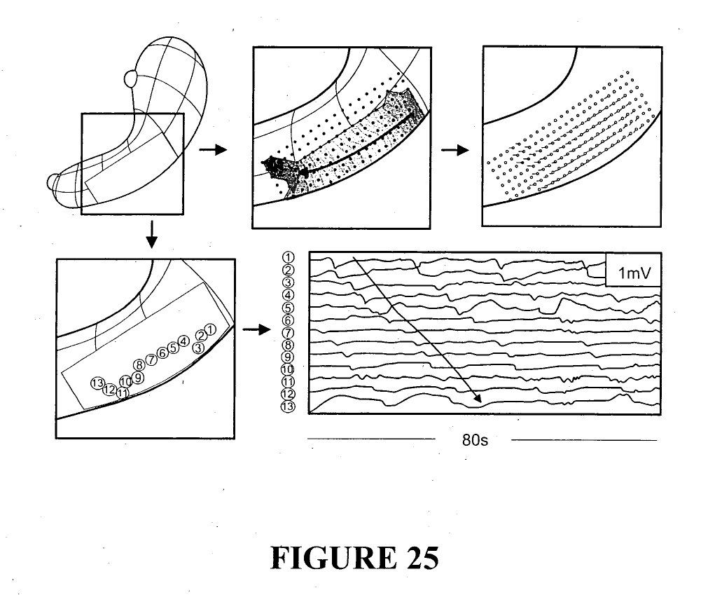

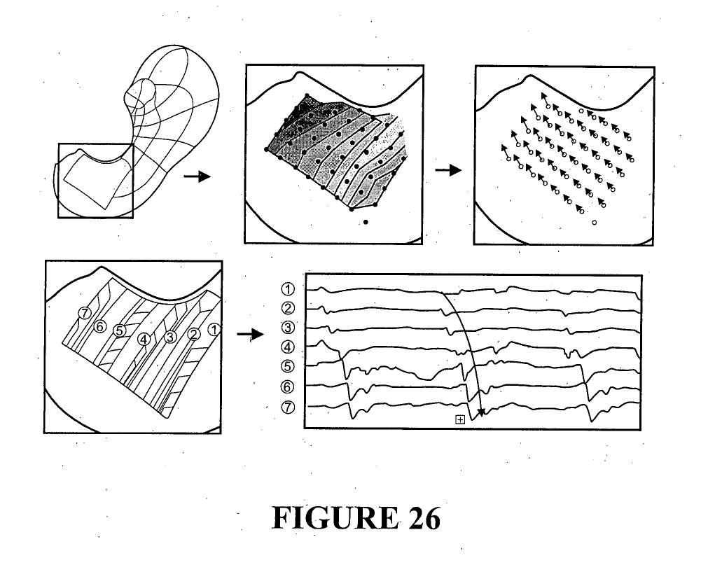

The regional velocities of slow wave propagation were averaged for all patients, and the result was mapped onto a 3D stomach model to demonstrate a generic visualization. Four activation time and velocity field maps of pacemaker activity, together with the representative gastric electrogram recordings used to create these spatial representations, are shown in FIGS. 23 to 26.

FIGS. 23a to 26a show stomach models showing the PCB placement. FIGS. 23b to 26c show individual electrode positions. FIGS. 23c to 26c are isochronal maps. FIGS. 23d to 26d are velocity maps. FIGS. 23e to 26e show representative gastric electrogram recordings from the electrodes used to create the spatial representations.

Greater than two simultaneous propagating wavefronts were observed in all patients, and between three and four simultaneous waves were observed in several cases.

### Example 3

### FEVT Activation Time Marking

Slow wave recordings of GI electrical activity were undertaken during surgery in pigs. Recordings were taken with both a high SNR 48 electrode array (resin-embedded, shielded, silver electrodes) and from a lower SNR electrode array (flexible PCBs; unshielded), from the anterior porcine gastric corpus. One 180 second representative data segment was selected from each of five animals: two segments from the high SNR array and three from the low SNR array. Unipolar recordings were acquired from the electrodes via the ActiveTwo System, at a recording frequency of 512 Hz. The common mode sense electrode was placed on the lower abdomen, and the right leg drive electrode on the hind leg. The electrodes array were connected to the ActiveTwo which was in turn connected to a notebook computer. The acquired signals were pre-processed by applying a second-order Butterworth digital band pass filter. The low frequency cutoff was set for 1 cpm ( 1/60 Hz); the high frequency cutoff was set to 60 cpm (1 Hz).

The slow wave ATs in each selected data segment were manually marked to provide a baseline for comparison. Within the electrode signal V(t), there are three dominant features of a slow wave event: (1) a small magnitude upstroke, immediately preceding (2) a fast, large magnitude, negative deflection (dV/dt˜=1 mV/s), followed by (3) a relatively long (5 s) plateau phase that decays slowly back to baseline. The fast negative-going transient corresponds with the depolarization wave front of the propagating slow wave, signaling the arrival of the slow wave at the recording electrode site. The point of most negative gradient during a slow wave was determined to be the AT.

Automated marking of the low SNR signals was carried out by the falling edge variable detection method. Some slow wave events exhibit a relatively fast recovery to baseline. This produces two large pulses in the transform detection signals, which can lead to erroneous double counting—the second mark in a set of two should not be marked. Such double-marking is precluded by imposing a criterion that distinct activation time events must be separated in time by a minimum value, termed the refractory period. Also, multiple slow wave events recorded by an electrode are not identical over time. For example, some pulses in a particular signal transform detection signals have larger amplitudes than the others. This amplitude difference can lead to missed detection of the smaller amplitude events. The FEVT algorithm implements a time-varying threshold (VT) to aid in the detection of ATs when recorded serosal waveforms may change over time.

Use was made of a falling-edge detector signal, E(t), to amplify the large-amplitude, high-frequency content associated only with negative deflections, suppressing positive-going transients in the process. It is formed by convolving the serosal electrical potential signal with an “edge-detector kernel” d_{Nedge}: E(t)=V(t)*d_{N}_{_{edge}} where * denotes the convolution operator. An edge-detector kernel (Sezan, Comput. Vis. Graph. Image Process. 49:36-51, 1990), was employed, which is formed from the convolution of a “smoother” with a “differencer”. N_{edge} defines the width of the kernel. A fixed value of N_{edge}=30, a 1-s wide kernel at fs=30 Hz, were chosen to correspond to the timescale of a typical large, negative transient. A falling edge (negative transient) in V(t) produces a positive deflection in E(t) (and vice-versa). When V(t) remains relatively constant, E(n) is approximately 0. Thus, E(t) is large and positive when V(t) contains a falling edge, and is negative for a rising edge. To help focus the slow wave detection algorithm on only the falling edges in V(t), the (element-wise) product of the smoothed detection signal S(t) was computed with the falling edge detection signal E(t), setting all negative values to zero. The resulting signal is termed the FEVT signal, F(t), which is thus summarized:

$$F ( t ) = \{ \begin{matrix}S ( t ) E ( t ) & if S ( t ) E ( t ) \geq 0 \\ 0 & if S ( t ) E ( t ) < 0.\end{matrix}$$

To avoid slight variations in the waveforms leading to some events escaping detection, the FEVT method incorporated a time-varying detection threshold. Specifically, the time-varying threshold is based on the running median of the absolute deviation for time t using a window of half-width τ_{HW} centered at t for the FEVT signal, F(t):

$$\hat{σ} ( t ) = M \{ \vert F ( t - {τ}_{HW} ) - \vert , … , \vert F ( t + {τ}_{HW} ) - \vert \} / 0.6745$$

where [symbol] is the sample mean of F(t) in the time range [t−τ_{HW}, t+τ_{HW}] and M{·} denotes the sample median, as before. The variable threshold was then defined as: Fₜₕᵣₑₛₕ=η×\hat{τ}(t), where η is a tunable parameter, as before. The moving median window was long enough to include the quiescent period in F(t) between the pulses of energy associated with the AT, but not so long that one slow wave can unduly influence the threshold defined for an event occurring much earlier or later. Values of 15, 30, and 45 s were used, which corresponds to about 1-2 full cycles 3 cpm gastric slow-wave waveform.

The FEVT method properly handled most problematic signals. For most electrodes, the FEVT detection algorithm succeeding in finding all ATs, without finding false positives. The overall performance of the FEVT algorithm was essentially invariant to the type of signal transform used when computing the FEVT signal. The FEVT detection signals contained large positive pulses corresponding to the negative-flanks of the corresponding electrode signal, while no such pulse was observed for positive-flank. The FEVT signals had a relatively high SNR. The time-varying threshold accommodates detection of ATs in an FEVT detection signal with a variable SNR. The FEVT algorithm was found suited to properly detect ATs in low SNR mucosally recorded signals.

### Example 4

### REGROUPS Cycle Clustering Method

Slow wave recordings were undertaken during surgery in pigs, and the recordings processed by the FEVT activation time marking method as described in Example 3. Recordings were taken with a low SNR array (flexible PCBs; unshielded), from the anterior porcine gastric corpus. Low SNR platforms were used because mucosal signals are typically of low SNR.

Four data sets (120 seconds duration) from four porcine subjects were selected because these segments represented a range of typical scenarios as follows:
Normal corpus propagation: Normally, gastric SWs propagate aborally as a transverse band (or ring) of activation, and consecutive wavefronts will be simultaneously detected by a large mapping array. A robust cycle partitioning algorithm must correctly determine which ATs belong to the distinct cycles, otherwise AT maps will be highly distorted and misleading. The first test case was from a corpus recordings on the greater curvature, featuring simultaneous, consecutive propagating wavefronts.
Normal pacemaker activity with peripheral region of quiescent tissue: Porcine SWs arise from a pacemaker area near the greater curvature of the mid-fundus; the upper and medial fundus are not activated. Robust analysis algorithms must correctly identify, the concentric propagation, while demarcating the inactive regions. The second test case was recorded from the porcine gastric pacemaker site.
Abnormal propagation: Periodic abnormal SW behaviors are observed during porcine HR gastric mapping often characterized by retrograde propagation and/or ectopic pacemaking. Robust analysis methods must correctly identify abnormal propagation patterns. The third and fourth test cases were selected from data sets exhibiting retrograde propagation and ectopic pacemaking, recorded from the upper corpus/distal fundus. Importantly, the latter three of these test cases also had patchy data quality, which results from suboptimal or obstructed electrode contact, or due to interfering signals (e.g., respiration artifacts).
Competing pacemakers/clashing wavefronts: When more than one region acts as a pacemaker, the multiple corresponding wavefronts generated by them will collide. Such dysrhythmic activity may correspond to clinically diagnosable conditions. Robust analysis methods must correctly identify that a single cycle contains multiple clashing wavefronts.

The REGROUPS algorithm works by clustering (x, y, t) points representing ATs into groups that represent independent cycles ((x, y) denotes the position of an electrode site (relative to an arbitrary reference), and t denotes an AT marked at that site). The algorithm is initialized by creating a master list of all marked ATs, and selecting the master seed electrode site in automated fashion (see below). A queue containing the (x; y) positions of nearby sites is established. A “nearby” site was defined as falling within a distance \sqrt{2}dₘᵢₙ of the seed electrode, where dₘᵢₙ denotes the minimum distance between the seed site and the closest site containing (at least) one AT. The factor of \sqrt{2} essentially defines a circular search radius (for a square lattice array) to include sites located diagonal to the seed. dₘᵢₙ is not necessarily equal to the inter-electrode spacing (although it often will be), enabling the algorithm to successfully “jump” across local patches of missing data.

REGROUPS also employs an iterative “flood fill” or “region growing” procedure. The first queue entry (electrode site) becomes the current seed, and all ATs at that site, AT(x; y; j) (where j=1, . . . , J indexes the marked ATs), are tested for membership. A point (x; y; t) in AT(x; y; j) is assigned membership to the cluster (or not) based on comparison to an estimated AT, Tₑₛₜ. If the difference is small enough, the AT which minimizes the estimate error is assigned membership to the cluster:

$${min}_{j} \vert AT ( x , y , j ) - {T}_{est} \leq Δ {t}_{max} ..$$

Once assigned, membership is never revoked. A point can be assigned membership to only one cluster (at most): Upon assignment, that (x; y; t) point is removed from master list of ATs so that is never tested again during the remainder of the clustering process. If the tested point is clustered, all of its nearby neighbors are added to the back of the queue, if they are not already in it. If the tested point is not clustered, it may be tested again for membership only after new cluster has initialized (a new activation time surface is calculated) at the next iteration. This restriction forces all wavefronts to be independent. Regardless, of whether any point was added to the cluster, the current seed is removed from the queue, and the next queue element becomes the current seed. Thus, the region in (x, y, t) space representing an independent cycle grows, terminating when the queue of nearby points becomes empty. At this stage, the cluster contains all ATs from one cycle. The same process is repeated anew to identify another independent cycle, starting with the next sequential AT marked at the master seed. Each iteration produces a cluster of (x, y, t) points, which represent the dynamics of an independent cycle. Points which are not assigned membership to any cluster are termed “orphans.”

A step is to implement a 2nd-order polynomial surface, T(x, y), to act as a continuously updating spatiotemporal filter, where: T(x,y)=p₁x²+p₂y²+p₃xy+p₄x+p₅y+p₆. Using only the (x, y, t) already in cluster, the vector of coefficients that defines the surface, p=[p1, p2, p3, p4, p5, p6], is computed using a previously described least-squares-fitting procedure: p=(A^{T}A)⁻¹At where A is a matrix whose rows are created using the (x, y) electrode positions of points already in the cluster: [x², y², xy, x, y, 1]; and t is a column vector containing the corresponding ATs marked at those electrode sites. Having solved for the vector of coefficients p that defines the polynomial surface, an estimate of the AT at a nearby site (xₙ, yₙ) can be obtained by simply extending the surface into that region: Tₑₛₜ=T(xₙ, yₙ). The coefficients describing the surface, p, are automatically updated every time another point is added to the cluster. Therefore, the data set at hand determines the form of the polynomial surface, making it substantially more robust and more widely-applicable for distinguishing independent cycles in a variety of SW behaviors. At least 6 points are required to obtain a fully determined system of equations, so prior to switching on the polynomial surface estimation, is computed as the mean of the ATs of the points already assigned membership in the cluster. In practice, we found the algorithm performs best when the polynomial surface estimation is switched on when the cluster size reaches a “critical mass” of at N_{crit}≧12 points, which is on the order of frac110 the total number of electrode sites on the recording platform (data not shown). If the critical mass is too small, then the surface was overfit to a small core of points, yielding a poor description of the propagation pattern across the entire electrode array. On the other hand, if the critical mass was too large, then the technique fails to utilize information about the velocity gradient at the wavefront boundary, which is critical for the success of the algorithm (other spatiotemporal filters may be introduced into the software to aid detection of different electrical patterns).

The outcome of clustering is dependent on the initial seed selection, particularly when the data quality is patchy (sparse). Seed selection was automated such that the seed was chosen to be at an electrode position (x, y)_{seed} which is typically embedded in a region providing the maximal density of information about the propagating wavefront:
For each electrode site, tally N(x, y), the total number of ATs detected at an electrode site location (x, y).
Compute the center of mass (CM) (x_{CM}, y_{CM}) using the entries of N(x, y):

$${x}_{CM} = \frac{{\sum }_{i} N ( {x}_{i} , {y}_{i} ) {x}_{i}}{{\sum }_{i} N ( {x}_{i} , {y}_{i} )}$$

where the sum is taken over all electrode sites, indexed by i. The y-coordinate y_{CM} is similarly computed.
Check if (x_{CM}, y_{CM}) corresponds to the coordinates of an electrode with marked ATs. If yes, then the seed is selected to be the CM. If not, move the seed to the closest electrode site meeting this condition. In practice, the seed is usually selected to be at the CM.

Isochronal slow wave activation maps were generated. Control and experimental arms were developed to compare completely automated versus completely manual results, starting from raw data and ending with AT maps. This approach therefore sought to validate the FEVT-REGROUPS-Automated-Isochronal-Mapping pipeline, to demonstrate real world practicability of the complete system:
experimental arm: ATs were identified via the FEVT method. The REGROUPS and automated isochronal mapping algorithms were applied to each FEVT auto-marked data set to identify the first 5 consecutive SW cycles.
control arm: ATs were manually assessed and marked by a fully blinded manual marker. ATs were manually marked at the apparent point of steepest negative slope. The resulting ATs were then manually partitioned to identify the first 5 consecutive SW cycles, and resultant isochronal maps generated. The manually generated maps were considered to be the standard for comparison.

Quantitative comparison: The automated results were quantitatively compared to the manually-derived results in terms of AT mapping a) area of coverage, and b) isochronal timing accuracy. The REGROUPS results showed strong similarity to the manual results with comparable isochronal intervals and orientations, comparable map coverage, and a high consistency between cycles. For normal pacemaker activity and peripheral quiescent region the REGROUPS results proved similar to the manual marking results with comparable isochronal intervals, orientations, and consistency between cycles, and similar spatial map coverage. For abnormal activity the manual maps and REGROUPS maps were highly comparable in terms of isochronal intervals and orientations. The REGROUPS consistently demonstrated slightly greater spatial coverage than the manual maps, extending proximally with a physiologically-consistent activation pattern.

### Example 5

### Gastric Pacing

Weaner pigs of either sex and of mean body weight of 36.1±2.6 kg were fasted overnight, before anaesthesia. The pigs were placed supine on a heating pad and laparotomy was performed.

Pacing was performed using a DS8000 stimulator (World Precision Instruments, Sarasota, Fla., USA) attached to two stainless-steel 23 g pacing needles (8 mm separation; 1.6 kΩ tissue resistance). All pacing protocols employed in this study were bipolar, involving constant current pulses of period 17 s-19 s, amplitudes of 2 to 4 mA, and a pulse width of 400 ms. Baseline recordings were taken prior to stimulation, and each protocol was evaluated for a duration of 5-20 minutes. The pacing needles were positioned in either the upper greater curvature, the distal antrum, or in the mid-corpus. The mid-corpus pacing site was employed to enable the study of entrained slow wave propagation in all directions from the stimulation site. The specific protocol used in each example study is described with the associated results.

HR mapping was performed using flexible printed circuit board PCB electrode arrays as described in Example 2 above. Signal analysis was as described in Example 2. Isochronal activation maps of selected propagation sequences were computed and velocity field maps for selected sequences were computed. Slow wave amplitudes were calculated. Where appropriate, slow wave parameters were averaged over multiple successive waves and expressed as means±s.d. Students' t-test was used to compare slow wave parameters, with a p-value<0.05 considered to be significant. An HR analysis allowed pacing outcomes to be evaluated at any pacing frequency, because the density of electrodes allows the slow wave propagation sequences to be tracked at superior spatial resolutions, allowing the spatial origin of pacing onset to be located precisely.

Gastric pacing was initiated at period 17 s, amplitude 4 mA, and pulse width 400 ms. The baseline slow wave frequency was 3.1±0.1 cpm, and pacing (3.52 cpm) successfully induced slow wave entrainment with a 1:1 relationship between each stimulus and entrained wave.

The foregoing describes the invention including embodiments and examples thereof, and alterations and modifications are intended to be incorporated in the scope hereof as defined in the accompanying claims.

1. A gastro-electrical activity mapping system comprising:
a catheter insertable through a natural orifice into the gastro-intestinal (GI) tract and comprising an array of electrodes for contacting an interior surface of a section of the GI tract to detect electrical potentials at multiple electrodes, and
a signal analysis and mapping system arranged to receive and process electrical signals from multiple electrodes of the array and spatio-temporally map wavefront propagation of GI smooth muscle electrical activity at said section of the GI tract, over a period of time.

2. A gastro-electrical activity mapping system according to claim 1 wherein the signal analysis and mapping system is arranged to spatially map and visually display to a user GI electrical activity in real time or near-real time.

3. A gastro-electrical activity mapping system according to claim 1 wherein the signal analysis and mapping system is arranged to map GI electrical activity as an activation time map of the GI electrical activity.

4. A gastro-electrical activity mapping system according to claims 1 wherein the signal analysis and mapping system is arranged to map GI electrical activity as a velocity map indicative of the direction and speed of the GI electrical activity.

5. A gastro-electrical activity mapping system according to claim 1 wherein the signal analysis and mapping system is arranged to map GI electrical activity as an amplitude map of the amplitude of the GI electrical activity.

6. A gastro-electrical activity mapping system according to claim 1 wherein the signal analysis and mapping system is arranged to map the GI electrical activity as a contour plot of the GI electrical activity.

7. A gastro-electrical activity mapping system according to claim 1 wherein the signal analysis and mapping system is arranged to map the GI electrical activity on an anatomical computer model of at least the section of the GI tract.

8. A gastro electrical activity mapping system according to claim 7 wherein the signal analysis and mapping system is arranged to map the GI electrical activity on a patient specific anatomical model of at least the section of the GI tract.

9-11. (canceled)

12. A gastro-electrical activity mapping system according to claim 1 wherein the signal analysis and mapping system is arranged to register the electrode array of the catheter on the anatomical model.

13-14. (canceled)

15. A gastro electrical activity mapping system according to claim 1 wherein the signal analysis and mapping system is arranged to map the GI electrical activity as an animation.

16. (canceled)

17. A gastro-electrical activity mapping system according to claim 1 wherein the signal analysis an processing system is arrange to analyse the GI electrical activity for events indicative of GI slow waves and then to cluster the detected events into groups each relating to a common GI slow wave base on temporal closeness.

18. A gastro-electrical activity mapping system according to claim 17 wherein the signal analysis an processing system is arrange to analyse the GI electrical activity for events indicative of slow waves by falling edge detection and a time varying threshold.

19. A gastro-electrical activity mapping system according to claim 18 wherein the falling edge detection comprises convolving the GI electrical activity with an edge detecting kernel.

20-22. (canceled)

23. A gastro-electrical activity mapping system according to claim 17 wherein the signal processing and mapping system is arrange to cluster the detected events by a region growing using polynomial surface estimate stabilization method.

24. A gastro-electrical activity mapping system according to claim 23 arrange to cluster detected events by selecting a master electrode, retrieving a list of events detected at the master electrode as master seeds, for each master seed creating a queue of events detected at nearby electrodes, and spatiotemporally filtering each queue of detected events.

25-32. (canceled)

33. A gastro-electrical activity mapping system according to claim 1 wherein the signal processing and mapping system is arranged to quantify averages of any one or more of GI electrical activity propagation directions, normal versus abnormal propagation, frequencies, regional stomach velocities, or amplitudes, an report an average figure and/or average map for a recording period.

34. A gastro-electrical activity mapping system according to claim 1 wherein the signal processing and mapping system is arrange to identify an report abnormal GI electrical activity.

35. A gastro-electrical activity mapping system according to claim 1 wherein the catheter comprises an electrode carrier carrying on an exterior surface the array of electrodes and expandable when in place to cause the electrodes to contact the interior surface of the GI tract.

36-39. (canceled)

40. A gastro-electrical activity mapping system according to claim 1 wherein the electrodes are point electrodes to indent the mucosa of the interior surface of the section of the GI tract to enhance electrical contact.

41. A gastro-electrical activity mapping system according to claim 1 wherein the catheter comprises between 3 and 10 rows of electrodes each space lengthwise of the catheter, an each row comprising between 3 and 10 electrodes.

42-47. (canceled)

48. A method for mapping GI electrical activity which comprises inserting a catheter through a natural orifice into the GI tract an causing an array of electrodes of the catheter to contact an interior surface of a section of the GI tract to detect electrical potentials at multiple electrodes, an receiving an spatio-temporally mapping wavefront propagation from the electrical signals GI electrical activity at said section of the GI tract, over a period of time.

49. A method according to claim 48 including mapping GI electrical activity as an activation time map.

50. A method according to claim 48 including mapping GI electrical activity as a velocity map indicative of the direction and speed of the GI electrical activity.

51. A method according to claim 48 including mapping GI electrical activity as an amplitude map indicative of the amplitude of the GI electrical activity.

52. A method according to claim 48 including mapping the GI electrical activity as a contour plot of the GI electrical activity.

53. A method according to claim 48 including mapping the GI electrical activity on an anatomical computer model of at least the section of the GI tract.

54. A method according to claim 53 including mapping the GI electrical activity on a patient-specific anatomical model of at least the section of the GI tract.

55. A method to claim 48 including analysing the GI electrical activity for events indicative of GI slow waves an clustering detected events into groups each relating to a common slow wave base on temporal closeness.

56. A method according to claim 55 including analysing the GI electrical activity for events indicative of slow waves by falling edge detection and a time varying threshold.

57-60. (canceled)

61. A method according to any of claim 55 including clustering detected events by a region growing using polynomial surface estimate stabilization method.

62-72. (canceled)

73. A catheter for mapping GI electrical activity, insertable through a natural orifice into the GI tract an comprising an array of sufficient point electrodes arrange to contact around and/or along an interior surface of a section of the GI tract to detect electrical potentials to enable mapping of electrical activity at said section of the GI tract.

74. A catheter according claim 73 which comprises an electrode carrier carrying on an exterior surface the array of electrodes an expandable when in place to cause the electrodes to contact the interior surface of the GI tract.

75. A catheter according to claim 74 wherein the expandable electrode carrier is expandable by fluid inflation.

76. A catheter according to claim 74 wherein the expandable electrode carrier comprises an expandable mesh.

77. A catheter according to claim 76 wherein the expandable mesh is resilient with a memory for its expanded condition.

78. (canceled)

79. A catheter according to claim 73 wherein the electrodes indent the mucosa of the interior surface of the section of the GI tract to enhance electrical contact.

80. (canceled)

81. A catheter according to claim 73 herein the array of electrodes comprises between 9 and 120 electrodes.

82. A catheter according to claim 73 wherein the electrodes comprise conductive protrusions of length between about 2 and about 5 mm.

83. A catheter according to claim 73 wherein the electrodes comprise conductive protrusions of length between about 2 and about 3 mm.

84. A catheter according to claim 73 wherein the electrodes comprise conductive protrusions of cross-sectional dimension between about 0.3 and about 3 mm.

85. A catheter according to claim 73 wherein the electrodes comprise conductive protrusions of cross-sectional dimension between about 0.5 and about 1.5 mm.

86. A catheter according to claim 73 wherein the electrodes comprise conductive protrusions of cross-sectional dimension between about 0.7 and about 1 mm.

87. A method for detecting GI slow wave activations in GI electrical activity which includes analysing the GI electrical activity for events indicative of GI slow waves and clustering detected events into groups each relating to a common slow wave based on temporal closeness.

88. A method according to claim 87 including analysing the GI electrical activity for events indicative of slow waves by falling edge detection and a time varying threshold.

89. A method according to claim 88 wherein the falling edge detection comprises convolving the GI electrical activity with an edge detecting kernel.

90. A method according to claim 88 wherein the time-varying threshold is calculated by a moving median window.

91-92. (canceled)

93. A method according to claim 88 including clustering detected events by a region growing using polynomial surface estimate stabilization method.

94. A method according to claim 93 including clustering detected events by selecting a master electrode, retrieving a list of detected events at the master electrode as master seeds, for each master seed creating a queue of events detected at nearby electrodes, and spatiotemporally filtering each queue of detected events.

95. A method according to claim 94 wherein including initialising a new cluster for each of the detected events at the master electrode.

96. A method according to claim 93 including counting the number of detected events in the cluster and generating a second order polynomial surface when the number of detected events in the cluster is greater than a critical mass.

97. A method according to claim 96 wherein the second order polynomial surface acts as the spatiotemporal filter.

98-102. (canceled)

103. A method for clustering detected GI slow wave events in GI electrical activity into groups each relating to a common slow wave base on temporal closeness, which comprises clustering detected events by a region growing using polynomial surface estimate stabilization method.

104. A method according to claim 103 including clustering detected events by selecting a master electrode, retrieving a list of detected events at the master electrode as master seeds, for each master seed creating a queue of events detected at nearby electrodes, an spatiotemporally filtering each queue of detected events.

105. A method according to claim 104 wherein including initialising a new cluster for each of the detected events at the master electrode.

106. A method according to claim 103 including counting the number of detected events in the cluster an generating a second order polynomial surface when the number of detected events in the cluster is greater than a critical mass.

107-112. (canceled)
